(19) United States
(12) Patent Application Publication
(10) Pub. No.: US 2023/0181447 A1
(43) Pub. Date: Jun. 15, 2023

(54) COSMETIC COMPOSITION COMPRISING CELLODEXTRINS

(71) Applicant: PFEIFER & LANGEN GMBH & CO. KG, Köln (DE)

(72) Inventor: Thomas HÄSSLER, Köln (DE)

(21) Appl. No.: 17/923,676

(22) PCT Filed: May 7, 2021
(86) PCT No.: PCT/EP2021/062167

§ 371 Date: Nov. 7, 2022

(30) Foreign Application Priority Data
May 8, 2020 (EP) 20173630.3

(51) Int. Cl. A61K 8/73; A61Q 19/10; A61Q 19/00

(52) U.S. Cl. CPC A61K 8/731; A61Q 19/10; A61Q 19/00; A61K 2800/412; A61K 2800/654

(57) ABSTRACT

The invention relates to a cosmetic composition comprising a cellodextrin component comprising a cellosaccharide selected from the group consisting of cellobiose, cellotriose, cellotetraose, cellopentaose, cellohexaose, celloheptaose, cellooctaose, cellononaose, cellodecaose, celloundecaose, cellododecaose, and combinations thereof; and (i) a lipoid component comprising one or more natural or synthetic lipoids; and/or (ii) a surfactant component comprising an anionic surfactant, cationic surfactant, amphoteric surfactant, nonionic surfactant; and/or (iii) a thickening component; and/or (iv) a pigment component; or any combination thereof; wherein at least a portion of said cellodextrin component is present within the cosmetic composition in undissolved form.

Priority is claimed of European patent application no. 20 173 630.3, filed on May 8, 2020.

### FIELD OF THE INVENTION

The invention relates to a cosmetic composition comprising a cellodextrin component comprising a cellosaccharide selected from the group consisting of cellobiose, cellotriose, cellotetraose, cellopentaose, cellohexaose, celloheptaose, cellooctaose, cellononaose, cellodecaose, celloundecaose, cellododecaose and combinations thereof; and (i) a lipoid component comprising one or more natural or synthetic lipoids; and/or (ii) a surfactant component comprising an anionic surfactant, cationic surfactant, amphoteric surfactant, nonionic surfactant, or any combination thereof; and/or (iii) a thickening component; and/or (iv) a pigment component; or any combination thereof; wherein at least a portion of said cellodextrin component is present within the cosmetic composition in undissolved form.

### BACKGROUND ART

In many cosmetic compositions, especially in soaps, wash lotions, peeling or scrub products, and tooth pastes raw materials such as polyethylene, polyurethane, polyamide, pol-yacrylates, and others are used, typically for functions such as exfoliating, cleansing, viscosity modification, optical appearance modification (e.g. white creamy optical effect) and others. Typically, these raw materials are present in form of small particles, also referred to as microplastic.

Such microplastic-containing cosmetic compositions have two major drawbacks: They are usually based on non-renewable resources and, especially when used in rinse-off cosmet-ics such as peelings, scrubs, tooth pastes or masks, are harmful for the environment. Environ-mental-friendly alternatives such as shells & kernels, fruits, seeds, minerals and others are known.

Cellodextrin (cellosaccharide)-containing compositions are known from the prior art. EP 1 930 012 relates to a cellooligosaccharide-containing composition for prevention or improve-ment of lifestyle-related diseases in the fields of foods and internal medicines.

However, the known alternatives are not satisfactory in every respect, especially when it comes to consumer acceptance. Many cosmetic compositions cannot be rinsed off easily and quickly, especially if they have a high content of hydrophobic ingredients such as lipoids. There is therefore a demand to improve washability of such cosmetic formulations. While washability can be improved by adding strong surfactants, such strong surfactants may have a detrimental effect on the skin, as they dissolve any oily substances, i.e. also those lipoids that are beneficial to the skin and naturally present in healthy skin. Further, many cosmetic compositions after use leave an unpleasant feel of the skin. For example, skin scrub creams (peelings) are used for mechanical exfoliation, i.e. removal of the oldest dead skin cells on the skin's outermost surface by abrasion. Several of these cosmetic compositions after use leave an unpleasant feel of the skin and thus suffer from poor consumer acceptance. There is a demand for cosmetic compositions having a low content of microplastic or do not contain microplastic at all and which have improved consumer acceptance, i.e. with regard to quick and easy washability and the effect on skin, compared to the cosmetic compositions known from the prior art.

Toothpastes generally include an abrasive material which is dispersed in a gel or paste base. Abrasives remove stains and plaque, as well as polish teeth. Common abrasives include calcium phosphates, alumina, calcium carbonate, and silica. Toothpaste must be abrasive enough to remove plaque and stains, but should not be so abrasive as to damage tooth enamel. Toothpastes are generally classified into two types: Opaque and transparent. Opaque toothpastes are usually white or colored. Such toothpastes usually contain calcium-based abrasives, in particular chalk. Gel toothpastes usually contain abrasive silica. Also known are multiphase toothpastes, which have an opaque phase and a transparent phase. Toothpastes, in particular-chalk based types, also contain thickening silica. Silica helps build up the viscosity. However, the thickening silica-based agent, especially at higher levels, may adversely affect the flavor delivery. This often compels formulators to dose an appropriate overage of the flavor. Since flavors are very expensive, even a slight reduction in flavor can be economically signifi-cant. Therefore, it is desirable to either reduce the level of thickening silica, or preferably, have no thickening silica. Toothpastes usually contain some polymers that are used for the delivery of flavor. One of the disadvantages of such polymers is that the unbalanced amount of such polymers can affect the spreadability of the paste. Furthermore, the paste becomes unstable after about a month of storage at elevated temperature. An arbitrary reduction in the level of such polymers cannot be a viable solution, as it may adversely affect delivery of flavor.

There is a demand for cosmetic compositions such as tooth pastes having optimized optical appearance, abrasive capacity, spreadability, storage stability and flavor delivery.

It is an object of the invention to provide cosmetic compositions having advantages compared to the cosmetic compositions of the prior art. The cosmetic compositions should be friendly to the environment and have improved consumer acceptance.

This object has been achieved by the subject-matter of the patent claims.

### SUMMARY OF THE INVENTION

The invention relates to a cosmetic composition comprising a cellodextrin component comprising a cellosaccharide selected from the group consisting of cellobiose, cellotriose, cellotetraose, cellopentaose, cellohexaose, celloheptaose, cellooctaose, cellononaose, cellodecaose, celloundecaose, cellododecaose and combinations thereof; and
(i) a lipoid component comprising one or more natural or synthetic lipoids; and/or
(ii) a surfactant component comprising an anionic surfactant, cationic surfactant, amphoteric surfactant, nonionic surfactant, or any combination thereof; and/or
(iii) a thickening component; and/or
(iv) a pigment component;
   wherein at least a portion of said cellodextrin component is present within the cosmetic composition in undissolved form.

The cosmetic compositions according to the invention are useful as skin and tooth cleansing compositions to be rinsed off with water after topical application to the skin and cleaning. Further, the cosmetic compositions according to the invention are useful as skin care composition to remain on the skin after topical application, e.g. exfoliating scrub soaps, body scrubs, peels (face peels, hand peels, foot peels), handwashing pastes, liquid soaps, shower gels, hair-removal compositions, face masks, and antiacne compositions and compositions suitable for keratin materials made from micronized biodegradable polymers and body lotions and cremes or powders and make-up. The compositions according to the invention are intended more particularly for application to human keratin materials, such as the skin (including the scalp), the nails and keratin fibers, and/or mucous membranes.

It has been surprisingly found that cosmetic compositions can be prepared containing a cellodextrin component, wherein at least a portion of said cellodextrin component is present within the cosmetic composition in undissolved form. It has been surprisingly found that such cosmetic compositions have excellent scrubbing properties, especially when the cellodextrin component that is present within the cosmetic composition in undissolved form forms particles of a suitable size to act as abrasive substance in mechanical exfoliation.

Further, it has been surprisingly found that the cosmetic compositions according to the invention have excellent consumer acceptance when compared with other cosmetic compositions of the same type not containing cellodextrin component. Still further, it has been surprisingly found that the cosmetic compositions according to the invention provide easy and also quick washability. Yet further, it has been surprisingly found that the cosmetic compositions according to the invention provide excellent skin effect/skin feel.

Furthermore, it has been surprisingly found that the cellodextrin component that is present within the cosmetic composition in undissolved form can provide the cosmetic composition with a white, creamy optical appearance, especially when the cellodextrin component is present in finely dispersed form. As cellodextrin is biologically degradable, the present invention makes it possible to avoid synthetic polymers that otherwise would be harmful for the environment as they would enter the environment in form of microplastic.

### DETAILED DESCRIPTION OF THE INVENTION

A first aspect of the invention relates to a cosmetic composition comprising a cellodextrin component comprising a cellosaccharide selected from the group consisting of cellobiose, cellotriose, cellotetraose, cellopentaose, cellohexaose, and combinations thereof;
(i) a lipoid component comprising one or more natural or synthetic lipoids; and/or
(ii) a surfactant component comprising an anionic surfactant, cationic surfactant, amphoteric surfactant, nonionic surfactant, or any combination thereof;
(iii) a thickening component; and/or
(iv) a pigment component;
   wherein at least a portion of said cellodextrin component is present within the cosmetic composition in undissolved form.

The cosmetic composition according to the invention comprises (i) lipoid component, and/or (ii) surfactant component, and/or (iii) thickening component, and/or (iv) pigment component. It is contemplated that the cosmetic composition comprises only a single one of components (i), (ii), (iii), and (iv); two of components (i), (ii), (iii), and (iv); three of components (i), (ii), (iii), and (iv); or all components (i), (ii), (iii), and (iv). Preferred combinations are selected from (i)+(ii), (i)+(iii), (i)+(iv), (ii)+(iii), (ii)+(iv), (iii)+(iv); (i)+(ii)+(iii), (i)+(ii)+(iv), (i)+(iii)+(iv), (ii)+(iii)+(iv); and (i)+(ii)+(iii)+(iv).

Unless expressly stated otherwise, all percentages are weight percent. For the purpose of the specification, unless expressly stated otherwise, “essentially consisting of” preferably refers to a content of at least 95 wt.-%, more preferably at least 98 wt.-%, still more preferably at least 99.0 wt.-%. Unless expressly stated otherwise, all norms such as EN ISO, DIN or ASTM referred to herein, are referred to in the version that is valid on Jan. 1, 2021.

For the purpose of the specification, cosmetic compositions encompass any composition that is intended to be applied to the human body for cleansing, beautifying, promoting attractiveness, or altering the appearance without affecting the body's structure or functions.

Cosmetic compositions according to the invention include various forms and product types. Cosmetic compositions according to the invention include solutions and dispersions, whereas the dispersions may be suspensions or emulsions, whereas the emulsions may be water-in-oil emulsions or oil-in-water emulsions and powdery forms. Any combinations are also contemplated according to the invention.

Non limiting examples of cosmetic compositions according to the invention include solutions (e.g. shampoos, body wash, hand cleansers such as foam soap and liquid soap, mas-caras, eye liners, colognes), creams/emulsions (e.g. hand moisturizers, make up, hair conditioners, sunscreens), lotions (e.g. facial moisturizers, leave-in hair conditioners and moisturiz-ing cleansers), suspensions (e.g. sunscreens, hand washes and shampoos), oint-ments/pastes, powders (e.g. aby powder, eye powder, foot powder), gels (hair products, body washes, shaving products and toothpastes), sticks, tablets, cakes, capsules (e.g. compact powder, eye shadow, cheek shadow cakes), exfoliating products (e.g. exfoliating scrub soap, body scrub, peel such as face peel, hand peel, foot peel, handwashing paste, tooth paste, hair-removal composition, face mask, or antiacne compositions).

The cosmetic composition according to the invention comprises a cellodextrin component comprising a cellosaccharide selected from the group consisting of cellobiose, cellotriose, cellotetraose, cellopentaose, cellohexaose, celloheptaose, cellooctaose, cellononaose, cellodecaose, celloundecaose, cellododecaose, and combinations thereof. Cellodextrins (some-times also referred to as “cellosaccharides”) are known to the skilled person. Cellodextrins are classified by the degree of polymerization (DP) which indicates the number of linked glucose monomers it contains, wherein each glucose monomer is linked via a beta-1,4 glycosidic bond; cellobiose (DP=2), cellotriose (DP=3), cellotetraose (DP=4), cellopentaose (DP=5), cellohexaose (DP=6), celloheptaose (DP=7), cellooctaose (DP=8), cellononaose (DP=9), cellodecaose (DP=10), celloundecaose (DP=11), cellododecaose (DP=12).

In preferred embodiments of the cosmetic composition according to the invention, the total content of the cellodextrin component is at least 0.01 wt.-%, or at least 0.1 wt.-%, or at least 1.0 wt.-%, preferably at least 5.0 wt.-%, more preferably at least 10 wt.-%, still more preferably at least 15 wt.-%, yet more preferably at least 20 wt.-%, even more preferably at least 25 wt.-%, yet more preferably at least 30 wt.-%, and most preferably at least 35 wt.-%, in each case relative to the total weight of the cosmetic composition.

In preferred embodiments of the cosmetic composition according to the invention, the weight content of the cellodextrin component is at most 45 wt.-%, or at most 42.5 wt.-%, or at most 40 wt.-%, or at most 37.5 wt.-%, or at most 35 wt.-%, or at most 32.5 wt.-%, or at most 30 wt.-%, or at most 27.5 wt.-%, or at most 25 wt.-%, or at most 22.5 wt.-%, in each case relative to the total weight of the cosmetic composition.

In a preferred embodiment, the cellodextrin component comprises or essentially consists of cellobiose (D=2).

In preferred embodiments of the cosmetic composition according to the invention, the weight content of cellobiose is at least at least 0.01 wt.-%, or at least 0.05 wt.-%, or at least 0.1 wt.-%, or at least 0.5 wt.-%, or at least 1.0 wt.-%, or at least 2.0 wt.-%, or at least 3.0 wt.-%, or at least 4.0 wt.-%, or at least 5.0 wt.-%, or at least 7.5 wt.-%, or at least 10 wt.-%, or at least 12.5 wt.-%, or at least 15 wt.-%, or at least 17.5 wt.-%, or at least 20 wt.-%, in each case relative to the total weight of the cosmetic composition.

In preferred embodiments of the cosmetic composition according to the invention, the weight content of cellobiose is at most 45 wt.-%, or at most 42.5 wt.-%, or at most 40 wt.-%, or at most 37.5 wt.-%, or at most 35 wt.-%, or at most 32.5 wt.-%, or at most 30 wt.-%, or at most 27.5 wt.-%, or at most 25 wt.-%, or at most 22.5 wt.-%, in each case relative to the total weight of the cosmetic composition.

In preferred embodiments of the cosmetic composition according to the invention, the weight content of cellobiose is at least 10 wt.-%, or at least 20 wt.-%, at least 30 wt.-%, or at least 40 wt.-%, at least 50 wt.-%, or at least 60 wt.-%, at least 70 wt.-%, or at least 80 wt.-%, at least 90 wt.-%, or at least 95 wt.-%, in each case relative to the total weight of the cellodextrin component.

In preferred embodiments of the cosmetic composition according to the invention, the weight content of cellobiose is at most 90 wt.-%, or at most 80 wt.-%, at most 70 wt.-%, or at most 60 wt.-%, at most 50 wt.-%, or at most 40 wt.-%, at most 30 wt.-%, or at most 20 wt.-%, at most 10 wt.-%, or at most 5.0 wt.-%, in each case relative to the total weight of the cellodextrin component.

In a preferred embodiment, the cellodextrin component comprises or essentially consists of cellotriose (DP=3).

In preferred embodiments of the cosmetic composition according to the invention, the weight content of cellotriose is at least at least 0.01 wt.-%, or at least 0.05 wt.-%, or at least 0.1 wt.-%, or at least 0.5 wt.-%, or at least 1.0 wt.-%, or at least 2.0 wt.-%, or at least 3.0 wt.-%, or at least 4.0 wt.-%, or at least 5.0 wt.-%, or at least 7.5 wt.-%, or at least 10 wt.-%, or at least 12.5 wt.-%, or at least 15 wt.-%, or at least 17.5 wt.-%, or at least 20 wt.-%, in each case relative to the total weight of the cosmetic composition.

In preferred embodiments of the cosmetic composition according to the invention, the weight content of cellotriose is at most 45 wt.-%, or at most 42.5 wt.-%, or at most 40 wt.-%, or at most 37.5 wt.-%, or at most 35 wt.-%, or at most 32.5 wt.-%, or at most 30 wt.-%, or at most 27.5 wt.-%, or at most 25 wt.-%, or at most 22.5 wt.-%, in each case relative to the total weight of the cosmetic composition.

In preferred embodiments of the cosmetic composition according to the invention, the weight content of cellotriose is at least 10 wt.-%, or at least 20 wt.-%, at least 30 wt.-%, or at least 40 wt.-%, at least 50 wt.-%, or at least 60 wt.-%, at least 70 wt.-%, or at least 80 wt.-%, at least 90 wt.-%, or at least 95 wt.-%, in each case relative to the total weight of the cellodextrin component.

In preferred embodiments of the cosmetic composition according to the invention, the weight content of cellotriose is at most 90 wt.-%, or at most 80 wt.-%, at most 70 wt.-%, or at most 60 wt.-%, at most 50 wt.-%, or at most 40 wt.-%, at most 30 wt.-%, or at most 20 wt.-%, at most 10 wt.-%, or at most 5.0 wt.-%, in each case relative to the total weight of the cellodextrin component.

In a preferred embodiment, the cellodextrin component comprises or essentially consists of cellotetraose (DP=4).

In preferred embodiments of the cosmetic composition according to the invention, the weight content of cellotetraose is at least at least 0.01 wt.-%, or at least 0.05 wt.-%, or at least 0.1 wt.-%, or at least 0.5 wt.-%, or at least 1.0 wt.-%, or at least 2.0 wt.-%, or at least 3.0 wt.-%, or at least 4.0 wt.-%, or at least 5.0 wt.-%, or at least 7.5 wt.-%, or at least 10 wt.-%, or at least 12.5 wt.-%, or at least 15 wt.-%, or at least 17.5 wt.-%, or at least 20 wt.-%, in each case relative to the total weight of the cosmetic composition.

In preferred embodiments of the cosmetic composition according to the invention, the weight content of cellotetraose is at most 45 wt.-%, or at most 42.5 wt.-%, or at most 40 wt.-%, or at most 37.5 wt.-%, or at most 35 wt.-%, or at most 32.5 wt.-%, or at most 30 wt.-%, or at most 27.5 wt.-%, or at most 25 wt.-%, or at most 22.5 wt.-%, in each case relative to the total weight of the cosmetic composition.

In preferred embodiments of the cosmetic composition according to the invention, the weight content of cellotetraose is at least 10 wt.-%, or at least 20 wt.-%, at least 30 wt.-%, or at least 40 wt.-%, at least 50 wt.-%, or at least 60 wt.-%, at least 70 wt.-%, or at least 80 wt.-%, at least 90 wt.-%, or at least 95 wt.-%, in each case relative to the total weight of the cellodextrin component.

In preferred embodiments of the cosmetic composition according to the invention, the weight content of cellotetraose is at most 90 wt.-%, or at most 80 wt.-%, at most 70 wt.-%, or at most 60 wt.-%, at most 50 wt.-%, or at most 40 wt.-%, at most 30 wt.-%, or at most 20 wt.-%, at most 10 wt.-%, or at most 5.0 wt.-%, in each case relative to the total weight of the cellodextrin component.

In a preferred embodiment, the cellodextrin component comprises or essentially consists of cellopentaose (DP=5).

In preferred embodiments of the cosmetic composition according to the invention, the weight content of cellopentaose is at least at least 0.01 wt.-%, or at least 0.05 wt.-%, or at least 0.1 wt.-%, or at least 0.5 wt.-%, or at least 1.0 wt.-%, or at least 2.0 wt.-%, or at least 3.0 wt.-%, or at least 4.0 wt.-%, or at least 5.0 wt.-%, or at least 7.5 wt.-%, or at least 10 wt.-%, or at least 12.5 wt.-%, or at least 15 wt.-%, or at least 17.5 wt.-%, or at least 20 wt.-%, in each case relative to the total weight of the cosmetic composition.

In preferred embodiments of the cosmetic composition according to the invention, the weight content of cellopentaose is at most 45 wt.-%, or at most 42.5 wt.-%, or at most 40 wt.-%, or at most 37.5 wt.-%, or at most 35 wt.-%, or at most 32.5 wt.-%, or at most 30 wt.-%, or at most 27.5 wt.-%, or at most 25 wt.-%, or at most 22.5 wt.-%, in each case relative to the total weight of the cosmetic composition.

In preferred embodiments of the cosmetic composition according to the invention, the weight content of cellopentaose is at least 10 wt.-%, or at least 20 wt.-%, at least 30 wt.-%, or at least 40 wt.-%, at least 50 wt.-%, or at least 60 wt.-%, at least 70 wt.-%, or at least 80 wt.-%, at least 90 wt.-%, or at least 95 wt.-%, in each case relative to the total weight of the cellodextrin component.

In preferred embodiments of the cosmetic composition according to the invention, the weight content of cellopentaose is at most 90 wt.-%, or at most 80 wt.-%, at most 70 wt.-%, or at most 60 wt.-%, at most 50 wt.-%, or at most 40 wt.-%, at most 30 wt.-%, or at most 20 wt.-%, at most 10 wt.-%, or at most 5.0 wt.-%, in each case relative to the total weight of the cellodextrin component.

In a preferred embodiment, the cellodextrin component comprises or essentially consists of cellohexaose (DP=6).

In preferred embodiments of the cosmetic composition according to the invention, the weight content of cellohexaose is at least at least 0.01 wt.-%, or at least 0.05 wt.-%, or at least 0.1 wt.-%, or at least 0.5 wt.-%, or at least 1.0 wt.-%, or at least 2.0 wt.-%, or at least 3.0 wt.-%, or at least 4.0 wt.-%, or at least 5.0 wt.-%, or at least 7.5 wt.-%, or at least 10 wt.-%, or at least 12.5 wt.-%, or at least 15 wt.-%, or at least 17.5 wt.-%, or at least 20 wt.-%, in each case relative to the total weight of the cosmetic composition.

In preferred embodiments of the cosmetic composition according to the invention, the weight content of cellohexaose is at most 45 wt.-%, or at most 42.5 wt.-%, or at most 40 wt.-%, or at most 37.5 wt.-%, or at most 35 wt.-%, or at most 32.5 wt.-%, or at most 30 wt.-%, or at most 27.5 wt.-%, or at most 25 wt.-%, or at most 22.5 wt.-%, in each case relative to the total weight of the cosmetic composition.

In preferred embodiments of the cosmetic composition according to the invention, the weight content of cellohexaose is at least 10 wt.-%, or at least 20 wt.-%, at least 30 wt.-%, or at least 40 wt.-%, at least 50 wt.-%, or at least 60 wt.-%, at least 70 wt.-%, or at least 80 wt.-%, at least 90 wt.-%, or at least 95 wt.-%, in each case relative to the total weight of the cellodextrin component.

In preferred embodiments of the cosmetic composition according to the invention, the weight content of cellohexaose is at most 90 wt.-%, or at most 80 wt.-%, at most 70 wt.-%, or at most 60 wt.-%, at most 50 wt.-%, or at most 40 wt.-%, at most 30 wt.-%, or at most 20 wt.-%, at most 10 wt.-%, or at most 5.0 wt.-%, in each case relative to the total weight of the cellodextrin component.

In a preferred embodiment, the cellodextrin component comprises or essentially consists of celloheptaose (DP=7).

In preferred embodiments of the cosmetic composition according to the invention, the weight content of celloheptaose is at least at least 0.01 wt.-%, or at least 0.05 wt.-%, or at least 0.1 wt.-%, or at least 0.5 wt.-%, or at least 1.0 wt.-%, or at least 2.0 wt.-%, or at least 3.0 wt.-%, or at least 4.0 wt.-%, or at least 5.0 wt.-%, or at least 7.5 wt.-%, or at least 10 wt.-%, or at least 12.5 wt.-%, or at least 15 wt.-%, or at least 17.5 wt.-%, or at least 20 wt.-%, in each case relative to the total weight of the cosmetic composition.

In preferred embodiments of the cosmetic composition according to the invention, the weight content of celloheptaose is at most 45 wt.-%, or at most 42.5 wt.-%, or at most 40 wt.-%, or at most 37.5 wt.-%, or at most 35 wt.-%, or at most 32.5 wt.-%, or at most 30 wt.-%, or at most 27.5 wt.-%, or at most 25 wt.-%, or at most 22.5 wt.-%, in each case relative to the total weight of the cosmetic composition.

In preferred embodiments of the cosmetic composition according to the invention, the weight content of celloheptaose is at least 10 wt.-%, or at least 20 wt.-%, at least 30 wt.-%, or at least 40 wt.-%, at least 50 wt.-%, or at least 60 wt.-%, at least 70 wt.-%, or at least 80 wt.-%, at least 90 wt.-%, or at least 95 wt.-%, in each case relative to the total weight of the cellodextrin component.

In preferred embodiments of the cosmetic composition according to the invention, the weight content of celloheptaose is at most 90 wt.-%, or at most 80 wt.-%, at most 70 wt.-%, or at most 60 wt.-%, at most 50 wt.-%, or at most 40 wt.-%, at most 30 wt.-%, or at most 20 wt.-%, at most 10 wt.-%, or at most 5.0 wt.-%, in each case relative to the total weight of the cellodextrin component.

In a preferred embodiment, the cellodextrin component comprises or essentially consists of cellooctaose (DP=8).

In preferred embodiments of the cosmetic composition according to the invention, the weight content of cellooctaose is at least at least 0.01 wt.-%, or at least 0.05 wt.-%, or at least 0.1 wt.-%, or at least 0.5 wt.-%, or at least 1.0 wt.-%, or at least 2.0 wt.-%, or at least 3.0 wt.-%, or at least 4.0 wt.-%, or at least 5.0 wt.-%, or at least 7.5 wt.-%, or at least 10 wt.-%, or at least 12.5 wt.-%, or at least 15 wt.-%, or at least 17.5 wt.-%, or at least 20 wt.-%, in each case relative to the total weight of the cosmetic composition.

In preferred embodiments of the cosmetic composition according to the invention, the weight content of cellooctaose is at most 45 wt.-%, or at most 42.5 wt.-%, or at most 40 wt.-%, or at most 37.5 wt.-%, or at most 35 wt.-%, or at most 32.5 wt.-%, or at most 30 wt.-%, or at most 27.5 wt.-%, or at most 25 wt.-%, or at most 22.5 wt.-%, in each case relative to the total weight of the cosmetic composition.

In preferred embodiments of the cosmetic composition according to the invention, the weight content of cellooctaose is at least 10 wt.-%, or at least 20 wt.-%, at least 30 wt.-%, or at least 40 wt.-%, at least 50 wt.-%, or at least 60 wt.-%, at least 70 wt.-%, or at least 80 wt.-%, at least 90 wt.-%, or at least 95 wt.-%, in each case relative to the total weight of the cellodextrin component.

In preferred embodiments of the cosmetic composition according to the invention, the weight content of cellooctaose is at most 90 wt.-%, or at most 80 wt.-%, at most 70 wt.-%, or at most 60 wt.-%, at most 50 wt.-%, or at most 40 wt.-%, at most 30 wt.-%, or at most 20 wt.-%, at most 10 wt.-%, or at most 5.0 wt.-%, in each case relative to the total weight of the cellodextrin component.

In a preferred embodiment, the cellodextrin component comprises or essentially consists of cellononaose (DP=9).

In preferred embodiments of the cosmetic composition according to the invention, the weight content of cellononaose is at least at least 0.01 wt.-%, or at least 0.05 wt.-%, or at least 0.1 wt.-%, or at least 0.5 wt.-%, or at least 1.0 wt.-%, or at least 2.0 wt.-%, or at least 3.0 wt.-%, or at least 4.0 wt.-%, or at least 5.0 wt.-%, or at least 7.5 wt.-%, or at least 10 wt.-%, or at least 12.5 wt.-%, or at least 15 wt.-%, or at least 17.5 wt.-%, or at least 20 wt.-%, in each case relative to the total weight of the cosmetic composition.

In preferred embodiments of the cosmetic composition according to the invention, the weight content of cellononaose is at most 45 wt.-%, or at most 42.5 wt.-%, or at most 40 wt.-%, or at most 37.5 wt.-%, or at most 35 wt.-%, or at most 32.5 wt.-%, or at most 30 wt.-%, or at most 27.5 wt.-%, or at most 25 wt.-%, or at most 22.5 wt.-%, in each case relative to the total weight of the cosmetic composition.

In preferred embodiments of the cosmetic composition according to the invention, the weight content of cellononaose is at least 10 wt.-%, or at least 20 wt.-%, at least 30 wt.-%, or at least 40 wt.-%, at least 50 wt.-%, or at least 60 wt.-%, at least 70 wt.-%, or at least 80 wt.-%, at least 90 wt.-%, or at least 95 wt.-%, in each case relative to the total weight of the cellodextrin component.

In preferred embodiments of the cosmetic composition according to the invention, the weight content of cellononaose is at most 90 wt.-%, or at most 80 wt.-%, at most 70 wt.-%, or at most 60 wt.-%, at most 50 wt.-%, or at most 40 wt.-%, at most 30 wt.-%, or at most 20 wt.-%, at most 10 wt.-%, or at most 5.0 wt.-%, in each case relative to the total weight of the cellodextrin component.

In a preferred embodiment, the cellodextrin component comprises or essentially consists of cellodecaose (DP=10).

In preferred embodiments of the cosmetic composition according to the invention, the weight content of cellodecaose is at least at least 0.01 wt.-%, or at least 0.05 wt.-%, or at least 0.1 wt.-%, or at least 0.5 wt.-%, or at least 1.0 wt.-%, or at least 2.0 wt.-%, or at least 3.0 wt.-%, or at least 4.0 wt.-%, or at least 5.0 wt.-%, or at least 7.5 wt.-%, or at least 10 wt.-%, or at least 12.5 wt.-%, or at least 15 wt.-%, or at least 17.5 wt.-%, or at least 20 wt.-%, in each case relative to the total weight of the cosmetic composition.

In preferred embodiments of the cosmetic composition according to the invention, the weight content of cellodecaose is at most 45 wt.-%, or at most 42.5 wt.-%, or at most 40 wt.-%, or at most 37.5 wt.-%, or at most 35 wt.-%, or at most 32.5 wt.-%, or at most 30 wt.-%, or at most 27.5 wt.-%, or at most 25 wt.-%, or at most 22.5 wt.-%, in each case relative to the total weight of the cosmetic composition.

In preferred embodiments of the cosmetic composition according to the invention, the weight content of cellodecaose is at least 10 wt.-%, or at least 20 wt.-%, at least 30 wt.-%, or at least 40 wt.-%, at least 50 wt.-%, or at least 60 wt.-%, at least 70 wt.-%, or at least 80 wt.-%, at least 90 wt.-%, or at least 95 wt.-%, in each case relative to the total weight of the cellodextrin component.

In preferred embodiments of the cosmetic composition according to the invention, the weight content of cellodecaose is at most 90 wt.-%, or at most 80 wt.-%, at most 70 wt.-%, or at most 60 wt.-%, at most 50 wt.-%, or at most 40 wt.-%, at most 30 wt.-%, or at most 20 wt.-%, at most 10 wt.-%, or at most 5.0 wt.-%, in each case relative to the total weight of the cellodextrin component.

In a preferred embodiment, the cellodextrin component comprises or essentially consists of celloundecaose (DP=11).

In preferred embodiments of the cosmetic composition according to the invention, the weight content of celloundecaose is at least at least 0.01 wt.-%, or at least 0.05 wt.-%, or at least 0.1 wt.-%, or at least 0.5 wt.-%, or at least 1.0 wt.-%, or at least 2.0 wt.-%, or at least 3.0 wt.-%, or at least 4.0 wt.-%, or at least 5.0 wt.-%, or at least 7.5 wt.-%, or at least 10 wt.-%, or at least 12.5 wt.-%, or at least 15 wt.-%, or at least 17.5 wt.-%, or at least 20 wt.-%, in each case relative to the total weight of the cosmetic composition.

In preferred embodiments of the cosmetic composition according to the invention, the weight content of celloundecaose is at most 45 wt.-%, or at most 42.5 wt.-%, or at most 40 wt.-%, or at most 37.5 wt.-%, or at most 35 wt.-%, or at most 32.5 wt.-%, or at most 30 wt.-%, or at most 27.5 wt.-%, or at most 25 wt.-%, or at most 22.5 wt.-%, in each case relative to the total weight of the cosmetic composition.

In preferred embodiments of the cosmetic composition according to the invention, the weight content of celloundecaose is at least 10 wt.-%, or at least 20 wt.-%, at least 30 wt.-%, or at least 40 wt.-%, at least 50 wt.-%, or at least 60 wt.-%, at least 70 wt.-%, or at least 80 wt.-%, at least 90 wt.-%, or at least 95 wt.-%, in each case relative to the total weight of the cellodextrin component.

In preferred embodiments of the cosmetic composition according to the invention, the weight content of celloundecaose is at most 90 wt.-%, or at most 80 wt.-%, at most 70 wt.-%, or at most 60 wt.-%, at most 50 wt.-%, or at most 40 wt.-%, at most 30 wt.-%, or at most 20 wt.-%, at most 10 wt.-%, or at most 5.0 wt.-%, in each case relative to the total weight of the cellodextrin component.

In a preferred embodiment, the cellodextrin component comprises or essentially consists of cellododecaose (DP=12).

In preferred embodiments of the cosmetic composition according to the invention, the weight content of cellododecaose is at least at least 0.01 wt.-%, or at least 0.05 wt.-%, or at least 0.1 wt.-%, or at least 0.5 wt.-%, or at least 1.0 wt.-%, or at least 2.0 wt.-%, or at least 3.0 wt.-%, or at least 4.0 wt.-%, or at least 5.0 wt.-%, or at least 7.5 wt.-%, or at least 10 wt.-%, or at least 12.5 wt.-%, or at least 15 wt.-%, or at least 17.5 wt.-%, or at least 20 wt.-%, in each case relative to the total weight of the cosmetic composition.

In preferred embodiments of the cosmetic composition according to the invention, the weight content of cellododecaose is at most 45 wt.-%, or at most 42.5 wt.-%, or at most 40 wt.-%, or at most 37.5 wt.-%, or at most 35 wt.-%, or at most 32.5 wt.-%, or at most 30 wt.-%, or at most 27.5 wt.-%, or at most 25 wt.-%, or at most 22.5 wt.-%, in each case relative to the total weight of the cosmetic composition.

In preferred embodiments of the cosmetic composition according to the invention, the weight content of cellododecaose is at least 10 wt.-%, or at least 20 wt.-%, at least 30 wt.-%, or at least 40 wt.-%, at least 50 wt.-%, or at least 60 wt.-%, at least 70 wt.-%, or at least 80 wt.-%, at least 90 wt.-%, or at least 95 wt.-%, in each case relative to the total weight of the cellodextrin component.

In preferred embodiments of the cosmetic composition according to the invention, the weight content of cellododecaose is at most 90 wt.-%, or at most 80 wt.-%, at most 70 wt.-%, or at most 60 wt.-%, at most 50 wt.-%, or at most 40 wt.-%, at most 30 wt.-%, or at most 20 wt.-%, at most 10 wt.-%, or at most 5.0 wt.-%, in each case relative to the total weight of the cellodextrin component.

In preferred embodiments, the cosmetic composition according to the invention essentially comprises neither cellotriose, nor cellotetraose, nor cellopentaose, nor cellohexaose, nor celloheptaose, nor cellooctaose, nor cellononaose, nor cellodecaose, nor celloundecaose, nor cellododecaose.

At least a portion of the cellodextrin component which is present in the cosmetic composition according to the invention is present in undissolved form.

Suitable methods for determining undissolved constituents in liquid compositions are known to the skilled person. For example, the presence of an undissolved portion of the cellodextrin component in the cosmetic composition according to the invention may be detectable through the naked eye or by means of turbidity measurements or absorbance-based optical density measurements using a nephelometer, spectrophotometer or Near-Infrared (NIR) spec-trometer, e.g. using a Dencytee® Sensor by the Hamilton Company (e.g. Dencytee® Unit DN12-120 having a length of 120 mm).

In preferred embodiments of the cosmetic composition according to the invention, the portion of the cellodextrin component that is present in undissolved form amounts to at least 5.0 wt.-%, or at least 10 wt.-%, or at least 15 wt.-%, or at least 20 wt.-%, or at least 25 wt.-%, or at least 30 wt.-%, or at least 35 wt.-%, or at least 40 wt.-%, or at least 45 wt.-%, or at least 50 wt.-%, or at least 55 wt.-%, or at least 60 wt.-%, or at least 65 wt.-%, or at least 70 wt.-%, or at least 75 wt.-%, or at least 80 wt.-%, or at least 85 wt.-%, or at least 90 wt.-%, or at least 95 wt.-%, in each case of the total amount of the cellodextrin component that is contained in the cosmetic composition.

In preferred embodiments of the cosmetic composition according to the invention, the portion of the cellodextrin component that is present in dissolved form amounts to at least 5.0 wt.-%, or at least 10 wt.-%, or at least 15 wt.-%, or at least 20 wt.-%, or at least 25 wt.-%, or at least 30 wt.-%, or at least 35 wt.-%, or at least 40 wt.-%, or at least 45 wt.-%, or at least 50 wt.-%, or at least 55 wt.-%, or at least 60 wt.-%, or at least 65 wt.-%, or at least 70 wt.-%, or at least 75 wt.-%, or at least 80 wt.-%, or at least 85 wt.-%, or at least 90 wt.-%, or at least 95 wt.-%, in each case of the total amount of the cellodextrin component that is contained in the cosmetic composition.

Methods for the quantification of the amount of the cellodextrin component that is present in undissolved form are also known to the skilled person, e.g. high-pressure liquid chromatography coupled with refractive index detector (HPLC-RID), gas chromatography mass spectrometry (GC/MS) or quantitative proton (¹H) nuclear magnetic resonance spectroscopy.

Preferably, the cosmetic composition comprises a multitude of solid particles, wherein at least a portion of the multitude of solid particles comprises at least a portion of the cellodextrin component (in the following said at least portion of solid particles is also referred to as “cellodextrin-containing particles”).

Preferably, the solid particles which comprise at least a portion of the cellodextrin component (i.e. the cellodextrin-containing particles) essentially consist of the cellodextrin component.

Preferably, substantially all particles of the multitude of solid particles comprise at least a portion of the cellodextrin component, i.e. preferably all particles contained in the cosmetic composition according to the invention are cellodextrin-containing particles.

Preferably, substantially the total amount of the cellodextrin component which is comprised in the cosmetic composition is comprised in the multitude of solid particles (i.e. in the cellodextrin-containing particles).

In preferred embodiments of the cosmetic composition according to the invention, the particles within the multitude of solid particles, i.e. the cellodextrin-containing particles, have a D50(v) value of at least 0.001 μm, or at least 0.005 μm, or at least 0.01 μm, or at least 0.05 μm, or at least 0.1 μm, or at least 0.5 μm, or at least 1.0 μm, or at least 5.0 μm, or at least 10 μm, or at least 50 μm, or at least 100 μm, determined by laser diffraction analysis according to ISO 13320.

In preferred embodiments of the cosmetic composition according to the invention, the particles within the multitude of solid particles, i.e. the cellodextrin-containing particles, have a D50(v) value of at most 100 μm, or at most 50 μm, or at most 10 μm, or at most 5.0 μm, or at most 1.0 μm, or at most 0.5 μm, or at most 0.1 μm, or at most 0.05 μm, or at most 0.01 μm, or at most 0.005 μm, or at most 0.001 μm, determined by laser diffraction analysis according to ISO 13320.

In preferred embodiments of the cosmetic composition according to the invention, the particles within the multitude of solid particles, i.e. the cellodextrin-containing particles, have an
D10(v) value within the range of from 0.1 μm to 2 500 μm; preferably from 1 000 μm to 2 000 μm, or from 500 μm to 1 000 μm, or from 200 μm to 500 μm, or from 75 μm to 200 μm, or from 0.1 μm to 4 μm, or from 0.3 μm to 1.0 μm, or from 0.3 μm to 0.8 μm; and/or
D50(v) value within the range of from 0.1 μm to 2 500 μm; preferably from 1 000 μm to 2 000 μm, or from 500 μm to 1 000 μm, or from 200 μm to 500 μm, or from 75 μm to 200 μm, or from 0.1 μm to 4 μm, or from 0.3 μm to 1.0 μm, or from 0.3 μm to 0.8 μm; and/or
D90(v) value within the range of from 0.1 μm to 2 500 μm; preferably from 1 000 μm to 2 000 μm, or from 500 μm to 1 000 μm, or from 200 μm to 500 μm, or from 75 μm to 200 μm, or from 0.1 μm to 4 μm, or from 0.3 μm to 1.0 μm, or from 0.3 μm to 0.8 μm;
   determined by laser diffraction analysis according to ISO 13320.

Preferably, the particles within the multitude of solid particles, i.e. the cellodextrin-containing particles, exhibit a certain span of the particle size distribution, wherein the span is typically defined as: (span)=(D90−D10)/D50.

In preferred embodiments of the cosmetic composition according to the invention, the particles within the multitude of solid particles, i.e. the cellodextrin-containing particles, exhibit a span of at least 0.5, or at least 1.0, or at least 1.5, or at least 2.0, or at least 2.5, or at least 3.0, or at least 3.5, or at least 4.0, or at least 4.5, or at least 5.0, or at least 5.5, or at least 6.0, or at least 6.5, or at least 7.0, or at least 7.5, or at least 8.0, or at least 8.5, or at least 9.0, or at least 9.5, or at least 10, determined by laser diffraction analysis according to ISO 13 320.

In preferred embodiments of the cosmetic composition according to the invention, the particles within the multitude of solid particles, i.e. the cellodextrin-containing particles, exhibit a span of at most 10, or at most 9.5, or at most 9.0, or at most 8.5, or at most 8.0, or at most 7.5, or at most 7.0, or at most 6.5, or at most 6.0, or at most 5.5, or at most 5.0, or at most 4.5, or at most 4.0, or at most 3.5, or at most 3.0, or at most 2.5, or at most 2.0, or at most 1.5, or at most 1.0, determined by laser diffraction analysis according to ISO 13 320.

In preferred embodiments of the cosmetic composition according to the invention, at least 10 wt.-%, or at least 20 wt.-%, or at least 30 wt.-%, or at least 40 wt.-%, or at least 50 wt.-%, or at least 60 wt.-%, or at least 70 wt.-%, or at least 80 wt.-%, or at least 90 wt.-% of the particles within the multitude of solid particles, i.e. the cellodextrin-containing particles, have an average particle size, preferably expressed as D50(v) value, within the range of from 0.1 μm to 2 500 μm, determined by laser diffraction analysis according to ISO 13320.

In preferred embodiments of the cosmetic composition according to the invention, at most 90 wt.-%, or at most 80 wt.-%, or at most 70 wt.-%, or at most 60 wt.-%, or at most 50 wt.-%, or at most 40 wt.-%, or at most 30 wt.-%, or at most 20 wt.-%, or at most 10 wt.-% of the particles within the multitude of solid particles, i.e. the cellodextrin-containing particles, have an average particle size, preferably expressed as D50(v) value, within the range of from 0.1 μm to 2 500 μm, determined by laser diffraction analysis according to ISO 13320.

In preferred embodiments of the cosmetic composition according to the invention, at least 10 wt.-%, or at least 20 wt.-%, or at least 30 wt.-%, or at least 40 wt.-%, or at least 50 wt.-%, or at least 60 wt.-%, or at least 70 wt.-%, or at least 80 wt.-%, or at least 90 wt.-% of the particles within the multitude of solid particles, i.e. the cellodextrin-containing particles, have an average particle size, preferably expressed as D50(v) value, within the range of from 1 000 μm to 2 000 μm, determined by laser diffraction analysis according to ISO 13320.

In preferred embodiments of the cosmetic composition according to the invention, at most 90 wt.-%, or at most 80 wt.-%, or at most 70 wt.-%, or at most 60 wt.-%, or at most 50 wt.-%, or at most 40 wt.-%, or at most 30 wt.-%, or at most 20 wt.-%, or at most 10 wt.-% of the particles within the multitude of solid particles, i.e. the cellodextrin-containing particles, have an average particle size, preferably expressed as D50(v) value, within the range of from 1 000 μm to 2 000 μm, determined by laser diffraction analysis according to ISO 13320.

In preferred embodiments of the cosmetic composition according to the invention, at least 10 wt.-%, or at least 20 wt.-%, or at least 30 wt.-%, or at least 40 wt.-%, or at least 50 wt.-%, or at least 60 wt.-%, or at least 70 wt.-%, or at least 80 wt.-%, or at least 90 wt.-% of the particles within the multitude of solid particles, i.e. the cellodextrin-containing particles, have an average particle size, preferably expressed as D50(v) value, within the range of from 500 μm to 1 000 μm, determined by laser diffraction analysis according to ISO 13320.

In preferred embodiments of the cosmetic composition according to the invention, at most 90 wt.-%, or at most 80 wt.-%, or at most 70 wt.-%, or at most 60 wt.-%, or at most 50 wt.-%, or at most 40 wt.-%, or at most 30 wt.-%, or at most 20 wt.-%, or at most 10 wt.-% of the particles within the multitude of solid particles, i.e. the cellodextrin-containing particles, have an average particle size, preferably expressed as D50(v) value, within the range of from 500 μm to 1 000 μm, determined by laser diffraction analysis according to ISO 13 320.

In preferred embodiments of the cosmetic composition according to the invention, at least 10 wt.-%, or at least 20 wt.-%, or at least 30 wt.-%, or at least 40 wt.-%, or at least 50 wt.-%, or at least 60 wt.-%, or at least 70 wt.-%, or at least 80 wt.-%, or at least 90 wt.-% of the particles within the multitude of solid particles, i.e. the cellodextrin-containing particles, have an average particle size, preferably expressed as D50(v) value, within the range of from 200 μm to 500 μm, determined by laser diffraction analysis according to ISO 13320.

In preferred embodiments of the cosmetic composition according to the invention, at most 90 wt.-%, or at most 80 wt.-%, or at most 70 wt.-%, or at most 60 wt.-%, or at most 50 wt.-%, or at most 40 wt.-%, or at most 30 wt.-%, or at most 20 wt.-%, or at most 10 wt.-% of the particles within the multitude of solid particles, i.e. the cellodextrin-containing particles, have an average particle size, preferably expressed as D50(v) value, within the range of from 200 μm to 500 μm, determined by laser diffraction analysis according to ISO 13 320.

In preferred embodiments of the cosmetic composition according to the invention, at least 10 wt.-%, or at least 20 wt.-%, or at least 30 wt.-%, or at least 40 wt.-%, or at least 50 wt.-%, or at least 60 wt.-%, or at least 70 wt.-%, or at least 80 wt.-%, or at least 90 wt.-% of the particles within the multitude of solid particles, i.e. the cellodextrin-containing particles, have an average particle size, preferably expressed as D50(v) value, within the range of from 75 μm to 200 μm, determined by laser diffraction analysis according to ISO 13 320.

In preferred embodiments of the cosmetic composition according to the invention, at most 90 wt.-%, or at most 80 wt.-%, or at most 70 wt.-%, or at most 60 wt.-%, or at most 50 wt.-%, or at most 40 wt.-%, or at most 30 wt.-%, or at most 20 wt.-%, or at most 10 wt.-% of the particles within the multitude of solid particles, i.e. the cellodextrin-containing particles, have an average particle size, preferably expressed as D50(v) value, within the range of from 75 μm to 200 μm, determined by laser diffraction analysis according to ISO 13 320.

In preferred embodiments of the cosmetic composition according to the invention, at least 10 wt.-%, or at least 20 wt.-%, or at least 30 wt.-%, or at least 40 wt.-%, or at least 50 wt.-%, or at least 60 wt.-%, or at least 70 wt.-%, or at least 80 wt.-%, or at least 90 wt.-% of the particles within the multitude of solid particles, i.e. the cellodextrin-containing particles, have an average particle size, preferably expressed as D50(v) value, within the range (i) of from 0.1 μm to 4.0 μm; or (ii) of from 0.2 μm to 2.0 μm; or (iii) of from 0.3 μm to 1.0 μm; or (iv) of from 0.3 μm to 0.8 μm; determined by laser diffraction analysis according to ISO 13 320.

In preferred embodiments of the cosmetic composition according to the invention, at most 90 wt.-%, or at most 80 wt.-%, or at most 70 wt.-%, or at most 60 wt.-%, or at most 50 wt.-%, or at most 40 wt.-%, or at most 30 wt.-%, or at most 20 wt.-%, or at most 10 wt.-% of the particles within the multitude of solid particles, i.e. the cellodextrin-containing particles, have an average particle size, preferably expressed as D50(v) value, within the range (i) of from 0.1 μm to 4.0 μm; or (ii) of from 0.2 μm to 2.0 μm; or (iii) of from 0.3 μm to 1.0 μm; or (iv) of from 0.3 μm to 0.8 μm; determined by laser diffraction analysis according to ISO 13320.

The cosmetic composition according to the invention preferably also comprises a lipoid component comprising one or more natural or synthetic lipoids.

In preferred embodiments of the cosmetic composition according to the invention, the weight content of the lipoid component is at least 5 wt.-%, or at least 10 wt.-%, or at least 15 wt.-%, or at least 20 wt.-%, or at least 25 wt.-%, or at least 30 wt.-%, or at least 35 wt.-%, or at least 40 wt.-%, or at least 45 wt.-%, or at least 50 wt.-%, or at least 55 wt.-%, or at least 60 wt.-%, or at least 65 wt.-%, in each case relative to the total weight of the cosmetic composition.

In preferred embodiments of the cosmetic composition according to the invention, the weight content of the lipoid component is at most 95 wt.-%, or at most 90 wt.-%, or at most 85 wt.-%, or at most 80 wt.-%, or at most 75 wt.-%, or at most 70 wt.-%, in each case relative to the total weight of the cosmetic composition.

Preferably, the lipoid component comprises or essentially consists of a natural lipoid. Natural lipoids can be selected from lipids of plant origin or animal origin.

Preferred natural lipoids of plant origin are selected from the group consisting of amaranth oil, apricot kernel oil, argan oil, avocado oil, acai oil, babassu oil, baobab oil, bayberry wax, black cumin oil, black currant seed oil, borage seed oil, brazil nut oil, broccoli seed oil, cacao butter, calendula oil, camelina oil, camellia seed oil, carrot seed oil, castor oil, cedarwood seed oil, chamomile oil, chia oil, coconut oil, corn oil, cottonseed oil, cupuacu butter, cranberry seed oil, eucalyptus oil, evening primrose oil, frankincense oil, geranium oil, grape seed oil, groundnut oil, hazelnut oil, hemp oil, illipe butter, Japan wax, jasmine oil, jojoba oil, kokum butter, kukui nut oil, laurel oil, lavender oil, lemongrass oil, linseed oil, macadamia nut oil, mango butter, manketti oil, marula oil, meadowfoam seed oil, milk butter, milk thistle oil, moringa oil, mustard seed oil, olive oil, olus oil, palm oil, palm kernel oil, papaya seed oil, passionfruit seed oil, peach kernel oil, peanut oil, pecan nut oil, peppermint oil, perilla oil, pistachio nut oil, plum kernel oil, pomegranate oil, poppyseed oil, pumpkin seed oil, rapeseed oil, raspberry seed oil, rice bran oil, rose oil, rosehip oil, rosemary oil, sacha inchi oil, safflower oil, sea buckthorn pulp oil, sesame oil, shea butter, soybean oil, St. John's wort oil, sunflower seed oil, squalane oil, sweet almond oil, tall oil, tamanu oil, tea-tree oil, tigernut oil, thyme oil, tung oil, walnut oil, wheat germ oil, and combinations thereof. Hydrocarbon oils of natural origin are for example terpene hydrocarbons such as squalene and squalane.

Preferred natural lipoids of animal origin are marine oils selected from the group consisting of fish oil, whale oil, fish liver oil, and seal oil.

Preferably, the lipoid component comprises or essentially consists of a synthetic lipoid. Synthetic lipoids include but are not limited to
(i) synthetic oils based upon fuel including mineral oil such as white mineral oil, paraffin oil, petroleum jelly oil, petrolatum and the like; liquid paraffins and its derivatives include iso-paraffins (e.g. isododecane, isohexadecane, polydecene hydrogenate) and cycloparaf-fins;
(ii) synthetic fats based upon fuel such as paraffin;
(iii) synthetic silicone oils such as polysiloxanes and their derivatives comprising for example alkyl, alkoxyl or phenyl groups, dimethylpolysiloxane, cyclic silicones, methylphenylpolysiloxane, silicone-glycol copolymers and the like; examples of silicone oils include the polydimethylsiloxanes (dimethicone), amodimethicone, cyclomethicones such as cyclopentasiloxane and cyclohexasiloxane, amino bispropyl dimethicone, ami-nopropyl dimethicone, amodimethicone hydroxystearate, behenoxy-dimethicone, C₃₀₋₄₅ alkyl dimethicone, C₂₄₋₂₈ alkyl dimethicone, C₃₀₋₄₅ alkyl methicone, cetearyl methicone, cetyl dimethicone, dimethoxysilyl ethylenediaminopropyl dimethicone, hexyl methicone, hy-droxypropyldimethicone, stearamidopropyl dimethicone, stearoxy dimethicone, stearyl methicone, stearyl dimethicone and vinyl dimethicone;
(iv) synthetic silicone fats;
(v) biochemically synthesized oils; or
(vi) biochemically synthesized fats.

Preferably, the lipoid component comprises or essentially consists of a combination of at least two, or at least three, or at least four, or at least five, or at least six of said natural and/or synthetic lipoids.

The cosmetic composition according to the invention may contain a wax. Suitable waxes are the waxes typically used in cosmetic compositions, and may be of natural and/or synthetic origin. Examples of natural waxes are beeswax or cera alba, carnauba wax, cande-lilla wax, Japan wax, rice wax, waxes deriving from hydrogenated oils such as jojoba oil or sunflower or coconut oils, esters of long chain saturated fatty acids with long chain mono-alcohols or their glycerides, such as cetyl palmitate, cetyl stearate, palmitic and stearic triglyc-erides. Examples of mineral or synthetic waxes are lignite wax, microcrystalline wax, paraffin, ozokerite, ceresin, synthetic beeswax, lanolin and their esters with polyethylene glycols, polyethylene waxes, fatty acid esters having a melting point above 25° C., cetyl esters, polyamides. Silicone waxes may also be used.

In preferred embodiments, the cosmetic composition according to the invention comprises essentially no water.

In other preferred embodiments, the cosmetic composition according to the invention additionally comprises water.

Suitable methods for determining the water content are known to the skilled person, e.g. Karl-Fischer titration.

In preferred embodiments of the cosmetic composition according to the invention, the water content is at least 20 wt.-%, or at least 25 wt.-%, or at least 30 wt.-%, or at least 35 wt.-%, or at least 40 wt.-%, or at least 45 wt.-%, or at least 50 wt.-%, or at least 55 wt.-%, or at least 60 wt.-%, or at least 65 wt.-%, or at least 70 wt.-%, or at least 75 wt.-%, or at least 80 wt.-%, or at least 85 wt.-%, in each case relative to the total weight of the cosmetic composition.

In preferred embodiments of the cosmetic composition according to the invention, the water content is at most 85 wt.-%, or at most 80 wt.-%, or at most 75 wt.-%, or at most 70 wt.-%, or at most 65 wt.-%, or at most 60 wt.-%, or at most 55 wt.-%, or at most 50 wt.-%, or at most 45 wt.-%, or at most 40 wt.-%, or at most 35 wt.-%, or at most 30 wt.-%, or at most 25 wt.-%, or most 20 wt.-%, or most 15 wt.-%, or at most 10 wt.-%, or at most 5.0 wt.-%, or at most 1.0 wt.-%, or at most 0.5 wt.-%, in each case relative to the total weight of the cosmetic composition.

The cosmetic composition according to the invention may be a dispersion, an emulsion, a suspension, or any mixture thereof.

According to the IUPAC and for the purpose of the specification, the term “dispersion” or “colloidal dispersion” is defined as a system in which particles of colloidal size of any nature (e.g. solid, liquid or gas) are dispersed in a continuous phase of a different composition (or state) (https://doi.org/10.1351/goldbook.001174).

According to the IUPAC and for the purpose of the specification, the term “emulsion” is defined as fluid colloidal system in which liquid droplets and/or liquid crystals are dispersed in a liquid. An emulsion is denoted by the symbol O/W if the continuous phase is an aqueous solution and by W/O if the continuous phase is an organic liquid (an ‘oil’) (https://doi.org/10.1351/goldbook.E02065).

According to the IUPAC and for the purpose of the specification, the term “suspension” is defined as a liquid in which solid particles are dispersed (https://doi.org/10.1351/goldbook.S06198).

In preferred embodiments, the cosmetic composition according to the invention is a water-in-oil emulsion.

In other preferred embodiments, the cosmetic composition according to the invention is an oil-in-water emulsion.

The cosmetic composition according to the invention preferably comprises a surfactant component comprising an anionic surfactant, cationic surfactant, amphoteric surfactant, nonionic surfactant, or any combination thereof.

In preferred embodiments of the cosmetic composition according to the invention, the weight content of the surfactant component is at least 0.1 wt.-%, or at least 1.0 wt.-%, or at least 2.0 wt.-%, or at least 4.0 wt.-%, or at least 6.0 wt.-%, or at least 8.0 wt.-%, or at least 10 wt.-%, or at least 15 wt.-%, or at least 20 wt.-%, or at least 25 wt.-%, or at least 30 wt.-%, or at least 35 wt.-%, or at least 40 wt.-%, or at least 45 wt.-%, or at least 50 wt.-%, or at least 55 wt.-%, or at least 60 wt.-%, or at least 65 wt.-%, or at least 70 wt.-%, or at least 75 wt.-%, or at least 80 wt.-%, or at least 85 wt.-%, or at least 90 wt.-%, or at least 95 wt.-%, in each case relative to the total weight of the cosmetic composition.

In preferred embodiments of the cosmetic composition according to the invention, the weight content of the surfactant component is at most 1.0 wt.-%, or at most 2.0 wt.-%, 4.0 wt.-%, or at most 6.0 wt.-%, or at most 8.0 wt.-%, or at most 10 wt.-%, or at most 15 wt.-%, or at most 20 wt.-%, or at most 25 wt.-%, or at most 30 wt.-%, or at most 35 wt.-%, or at most 40 wt.-%, or at most 45 wt.-%, or at most 50 wt.-%, or at most 55 wt.-%, or at most 60 wt.-%, or at most 65 wt.-%, or at most 70 wt.-%, or at most 75 wt.-%, or at most 80 wt.-%, or at most 85 wt.-%, or at most 90 wt.-%, or at most 95 wt.-%, in each case relative to the total weight of the cosmetic composition.

In preferred embodiments of the cosmetic composition according to the invention, the surfactant component comprises or essentially consists of a surfactant selected from the group consisting of
anionic surfactants which are preferably selected from: sodium laureth sulfate, sodium dodecyl sulfate, sodium lauryl sulfate, ammonium lauryl sulfate, sulfosuccinates (e.g. dioctyl sulfosuccinate, disodium laureth sulfosuccinate, disodium PEG-5 laurylcitrate sulfosuccinate, diethylhexyl sodium sulfosuccinate, disodium ricinoleamido MEA-sulfosuccinate, disodium undecylenamido MEA-sulfosuccinate), acyl methyl taurates (e.g. sodium N-methyl lauroyl taurate), alkylbenzene sulfonates (e.g. dodecylbenzene sulfonate), acyl sarcosinates (e.g. sodium lauroyl sarcosinate), fatty glycerol ether sulfonates (e.g. sodium (3-lauryloxy)-2-hydroxypropane sulfonate), acyl isethionate (e.g. sodium lauroly isethionate), monoglyceride sulfates (e.g. sodium (3-lauroyloxy)-2-hydroxypropyl sulfate), propyl peptide condensates;
cationic surfactants which are preferably selected from: stearalkonium chlorides (benzyldimethyloctadecylammonium chloride), dicetyldimonium chloride, docosyltrimethylammonium chloride;
amphoteric surfactants which are preferably selected from: sodium lauraminopropionate, sodium cocoamphopropionate, cocamidopropyl betaines (e.g. lauramidopropyl betaine), capryl/capramidopropyl betaine, undecylenamidopropyl betaine, disodium cocoamphodiacetate, sodium cocoamphoacetate;
nonionic surfactants which are preferably selected from: lauramide, lauramide diethanolamine, lauramide oxide, stearamine oxide, poloxamers (e.g. Poloxamer 101, Poloxamer 124), PEG-7 glyceryl cocoate, glyceryl laurate, cocamide diethanolamine, cocamide monoethanolamine;
biosurfactants which are preferably selected from (i) glycolipids (carbohydrate-lipids) such as rhamnolipids, mannosylerythritol lipids, sophorolipids, cellobioselipids, and the like; (ii) phospholipids; (iii) polyol lipids; (iv) lipoproteins; (v) lipopeptides; (vi) ornithine lipids; (vii) neutral lipids; (viii) aminoacid lipids; (ix) exolipids; (x) liposan; (xi) siderolipids; (xii) protein polyamines diglycosyl diglycerides; (xiii) fimbriae;
   and combinations thereof.

In preferred embodiments, the cosmetic composition according to the invention additionally comprises an antioxidant.

Preferably, the antioxidant is selected from tocopherols. Other antioxidants that can be used in the cosmetic composition according to the invention include but are not limited to citric acid, gallic acid, esters of gallic acid, nordihydroguaiaretic acid, thioctic acid (lipoic acid), dihy-drolipoic acid, glycolic acid, butylated hydroxytoluene (BHT), butylated hydroxyanisole (BHA), vitamins (e.g. tocopherol, retinol, p-carotene, ascorbic acid) vitamin derivatives (e.g. retinyl acetate, retinyl palmitate, ascorbyl palmitate, magnesium ascorbyl phosphate, tocophoryl acetate), flavonoids, polyphenolic compounds, and the like.

In preferred embodiments, the cosmetic composition according to the invention additionally comprises one or more additives independently of one another selected from the group consisting of acids, fragrances, essential oils, dyes, pigments (e.g. a pigment component), solvents, fillers, thickening agents (e.g. a thickening component), sequestrants, chelating agents, UV absorbers, preservatives, emulsifiers, emollients, and humectants.

In preferred embodiment, the thickening agent comprises or essentially consists of
(i) a natural or synthetic gum, preferably selected from the group consisting of algin, sodium alginate, carrageenan, cellulose gum, guar gum, xanthan gum, karaya gum, gelatin, tragacanth gum, chitosan, acacia gum, and any combination thereof;
(ii) a cellulose ether, preferably selected from the group consisting of methylcellulose, ethylcellulose, hydroxyethyl cellulose, hydroxypropyl cellulose, hydroxypropyl methyl cellulose, carboxymethyl cellulose, and any combination thereof;
(iii) a carbomer (e.g., a crosslinked polyacrylic acid copolymer or homopolymer and copolymers of acrylic acid cross linked with a polyalkenyl polyether);
(iv) an acrylamide polymer, acrylic acid polymer or vinyl polymer, preferably selected from polyvinyl alcohol and polyvinylpyrrolidone;
(v) a polyamine or polyquaternary compound;
(vi) an ethylene oxide polymer;
   or any mixtures thereof.

In particularly preferred embodiments of the cosmetic composition according to the invention,
at least 50 wt.-%, preferably all of the particles within the multitude of solid particles, i.e. the cellodextrin-containing particles, which preferably essentially consist of the cellodextrin component, more preferably of cellobiose, have an average particle size, preferably expressed as D50(v) value, within the range of from 0.1 μm to 4.0 μm, or from 0.2 μm to 2.0 μm, or from 0.3 μm to 1.0 μm, or from 0.3 μm to 0.8 μm, determined by laser diffraction analysis according to ISO 13 320; and/or
the weight content of the surfactant component is at least 5.0 wt.-%, preferably at least 10 wt.-%, relative to the total weight of the cosmetic composition; and/or
the cosmetic composition additionally comprises water, wherein the water content is at least 80 wt.-%, relative to the total weight of the cosmetic composition.

In particularly preferred embodiments of the cosmetic composition according to the invention,
the cellodextrin component comprises or essentially consists of cellobiose; and/or
the cosmetic composition comprises solid particles comprising or essentially consisting of the cellodextrin component in undissolved form; and/or
the particles have an average particle size, preferably expressed as D50(v) value determined by laser diffraction analysis according to ISO 13320, of at most 100 μm, or at most 50 μm, or at most 10 μm, or at most 5.0 μm, or at most 1.0 μm, or at most 0.5 μm, or at most 0.1 μm, or at most 0.05 μm, or at most 0.01 μm, or at most 0.005 μm, or at most 0.001 μm; and/or
the weight content of the surfactant component is at least 0.1 wt.-%, preferably at least 1.0 wt.-%, more preferably at least 5.0 wt.-%, still more preferably at least 10 wt.-%, relative to the total weight of the cosmetic composition; and/or
the cosmetic composition additionally comprises water, wherein the water content is at least 20 wt.-%, preferably at least 40 wt.-%, more preferably at least 60 wt.-%, still more preferably at least 80 wt.-%, relative to the total weight of the cosmetic composition; and/or
the cosmetic composition is selected from liquid soap, foam soap, shower gel and hair shampoo.

For the purpose of the specification opaque means not transparent or translucent.

In particularly preferred embodiments of the cosmetic composition according to the invention,
at least 50 wt.-%, preferably all of the particles within the multitude of solid particles, i.e. the cellodextrin-containing particles, which preferably essentially consist of the cellodextrin component, more preferably of cellobiose, have an average particle size, preferably expressed as D50(v) value, within the range of from 1 000 μm to 2 000 μm, or from 500 μm to 1 000 μm, or from 200 μm to 500 μm, or from 75 μm to 200 μm, determined by laser diffraction analysis according to ISO 13320; and/or
the weight content of the lipoid component is at least 50 wt.-%, preferably 60 wt.-%; and/or
the total content of the cellodextrin component is at least 20 wt.-%; and/or
the portion of the cellodextrin component that is present in undissolved form amounts to at least 95 wt.-%, relative to the total amount of the cellodextrin component that is contained in the cosmetic composition; and/or
the cosmetic composition comprises essentially no water.

In particularly preferred embodiments of the cosmetic composition according to the invention,
the cellodextrin component comprises or essentially consists of cellobiose; and/or
the cosmetic composition comprises solid particles comprising or essentially consisting of the cellodextrin component in undissolved form; and/or
the particles have an average particle size, preferably expressed as D50(v) value determined by laser diffraction analysis according to ISO 13 320, of at least 0.001 μm, or at least 0.005 μm, or at least 0.01 μm, or at least 0.05 μm, or at least 0.1 μm, or at least 0.5 μm, or at least 1.0 μm, or at least 5.0 μm, or at least 10 μm, or at least 50 μm, or at least 100 μm; and/or
the weight content of the lipoid component is at least 0.1 wt.-%, preferably at least 1.0 wt.-%, more preferably at least 5.0 wt.-%, still more preferably at least 10 wt.-%, yet more preferably at least 20 wt.-%, even more preferably at least 30 wt.-%, most preferably at least 40 wt.-%, and in particular at least 50 wt.-%, relative to the total weight of the cosmetic composition; and/or
the cosmetic composition comprises essentially no water, or wherein the water content is at most 20 wt.-%, preferably at most 15 wt.-%, more preferably at most 10 wt.-%, still more preferably at most 5.0 wt.-%, relative to the total weight of the cosmetic composition; and/or
the cosmetic composition is selected from the group consisting of exfoliating scrub soaps, body scrubs, peels (face peels, hand peels, foot peels), handwashing pastes, liquid soaps, shower gels, hair-removal compositions, face masks, and antiacne compositions.

In preferred embodiments, the cosmetic composition according to the invention has an opacity of at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or at least 100%, determined by colorimeter measurements.

In preferred embodiments, the cosmetic composition according to the invention has an opacity of at most 95%, or at most 90%, or at most 85%, or at most 80%, or at most 75%, or at most 70%, or at most 65%, or at most 60%, or at most 55%, or at most 50%, or at most 45%, or at most 40%, or at most 35%, or at most 30%, or at most 25%, determined by colorimeter measurements.

In preferred embodiments, the cosmetic composition according to the invention has a turbidity of at least 5 NTU, or at least 10 NTU, or at least 20 NTU, or at least 40 NTU, or at least 60 NTU, or at least 80 NTU, or at least 100 NTU, or at least 200 NTU, or at least 400 NTU, or at least 600 NTU, or at least 800 NTU, or at least 1 000 NTU, determined by nephelometric analysis.

In preferred embodiments, the cosmetic composition according to the invention has a turbidity of at most 900 NTU, or at most 800 NTU, or at most 600 NTU or at most 400 NTU or at most 200 NTU or at most 100 NTU, or at most 80 NTU or at most 60 NTU or at most 40 NTU or at most 20 NTU or at most 10 NTU, determined by nephelometric analysis.

Preferably, the cellodextrin component that is present within the cosmetic composition in undissolved form causes the turbidity of the cosmetic composition according to the invention.

In preferred embodiments, the cosmetic composition according to the invention has an intensity of pearlescence, on a scale of 0 to 10, of at least 1, or at least 2, or at least 3, or at least 4, or at least 5, or at least 6, or at least 7, or at least 8, or at least 9, determined in a sensory test according to ISO 13299.

In preferred embodiments, the cosmetic composition according to the invention has an intensity of pearlescence, on a scale of 0 to 10, of at most 9, or at most 8, or at most 7, or at most 6, or at most 5, or at most 4, or at most 3, or at most 2, or at most 1, determined in a sensory test according to ISO 13299.

Preferably, the cellodextrin component that is present within the cosmetic composition in undissolved form causes the pearlescence of the cosmetic composition according to the invention.

In preferred embodiments, the cosmetic composition according to the invention has a dynamic viscosity of at least 10 mPa·s, or at least 20 mPa·s, or at least 40 mPa·s, or at least 80 mPa·s, or at least 100 mPa·s, or at least 200 mPa·s, or at least 400 mPa·s, or at least 600 mPa·s, or at least 800 mPa·s, or at least 1 000 mPa·s, determined according to ISO 3219.

In preferred embodiments, the cosmetic composition according to the invention has a dynamic viscosity of at most 1 000 mPa·s, or at most 800 mPa·s, or at most 600 mPa·s, or at most 400 mPa·s, or at most 200 mPas, or at most 100 mPas, or at most 80 mPas, or at most 40 mPas, or at most 20 mPas, or at most 10 mPas, determined according to ISO 3219.

In preferred embodiments, the cosmetic composition according to the invention has a dynamic viscosity of at least 2 000 mPas, or at least 4 000 mPas, or at least 6 000 mPas, or at least 8 000 mPas, or at least 10 000 mPas, or at least 11 000 mPas, or at least 12 000 mPas, or at least 13 000 mPas, or at least 14 000 mPas, or at least 15 000 mPas, determined according to ISO 3219.

In preferred embodiments, the cosmetic composition according to the invention has a dynamic viscosity of at most 100 000 000 mPas, or at most 10 000 000 mPas, or at most 5 000 000 mPas, or at most 1 000 000 mPas, or at most 500 000 mPas, or at most 250 000 mPas, or at most 100 000 mPas, or at most 50 000 mPas, or at most 25 000 mPas, or at most 15 000 mPas, or at most 14 000 mPas, or at most 13 000 mPas, or at most 12 000 mPas, or at most 11 000 mPas, or at most 10 000 mPas, or at most 8 000 mPas, or at most 4 000 mPas, or at most 2 000 mPa determined according to ISO 3219.

In preferred embodiments, the cosmetic composition according to the invention comprises a thickening component. The thickening component is generally used to control or mod-ify the viscosity of the cosmetic composition. This embodiment is particularly relevant when the cosmetic composition according to the invention is e.g. a tooth paste.

In preferred embodiments of the cosmetic composition according to the invention, the weight content of the thickening component is at least 0.01 wt.-%, or at least 0.025 wt.-%, or at least 0.05 wt.-%, or at least 0.075 wt.-%, or at least 0.1 wt.-%, or at least 0.25 wt.-%, or at least 0.5 wt.-%, or at least 0.75 wt.-%, or at least 1.0 wt.-%, or at least 1.25 wt.-%, or at least 1.5 wt.-%, or at least 1.75 wt.-%, or at least 2.0 wt.-%, or at least 2.25 wt.-%, or at least 2.5 wt.-%, or at least 2.75 wt.-%, or at least 3.0 wt.-%, or at least 3.25 wt.-%, or at least 3.5 wt.-%, or at least 3.75 wt.-%, or at least 4.0 wt.-%, or at least 4.25 wt.-%, or at least 4.5 wt.-%, or at least 4.75 wt.-%, or at least 5.0 wt.-%, in each case relative to the total weight of the cosmetic composition.

In preferred embodiments of the cosmetic composition according to the invention, the weight content of the thickening component is at most 2.5 wt.-%, or at most 3.0 wt.-%, or at most 3.5 wt.-%, or at most 4.0 wt.-%, or at most 4.5 wt.-%, or at most 5.0 wt.-%, or at most 6.5 wt.-%, or at most 7.0 wt.-%, or at most 7.5 wt.-%, or at most 8.0 wt.-%, or at most 9.0 wt.-%, or at most 10 wt.-%, or at most 12.5 wt.-%, or at most 15 wt.-%, or at most 17.5 wt.-%, or at most 20 wt.-%, or at most 22.5 wt.-%, or at most 25 wt.-%, in each case relative to the total weight of the cosmetic composition.

Preferably, the thickening component comprises or essentially consists of
(i) a natural or synthetic gum, preferably selected from the group consisting of algin, sodium alginate, carrageenan, cellulose gum, guar gum, xanthan gum, karaya gum, gelatin, tragacanth gum, chitosan, acacia gum, and any combination thereof;
(ii) a cellulose ether, preferably selected from the group consisting of methylcellulose, ethylcellulose, hydroxyethyl cellulose, hydroxypropyl cellulose, hydroxypropyl methyl cellulose, carboxymethyl cellulose, and any combination thereof;
(iii) a carbomer (e.g., a crosslinked polyacrylic acid copolymer or homopolymer and copolymers of acrylic acid cross linked with a polyalkenyl polyether);
(iv) an acrylamide polymer, acrylic acid polymer or vinyl polymer, preferably selected from polyvinyl alcohol and polyvinylpyrrolidone;
(v) a polyamine or polyquarternary compound;
(vi) an ethylene oxide polymer;
   and any mixtures thereof.

In preferred embodiments, the cosmetic composition according to the invention comprises a pigment component. The pigment component is generally used to provide chromatic modifications to the cosmetic composition, e.g. to make-ups. This embodiment is particularly relevant when the cosmetic composition according to the invention is a make-up, especially a powdery make-up selected from loose powders and compact powders, face powders, founda-tions, blushers, and eyeshadows.

In preferred embodiments of the cosmetic composition according to the invention, the weight content of the pigment component is at least 0.01 wt.-%, or at least 0.025 wt.-%, or at least 0.05 wt.-%, or at least 0.075 wt.-%, or at least 0.1 wt.-%, or at least 0.25 wt.-%, or at least 0.5 wt.-%, or at least 0.75 wt.-%, or at least 1.0 wt.-%, or at least 1.25 wt.-%, or at least 1.5 wt.-%, or at least 1.75 wt.-%, or at least 2.0 wt.-%, or at least 2.25 wt.-%, or at least 2.5 wt.-%, or at least 2.75 wt.-%, or at least 3.0 wt.-%, or at least 3.25 wt.-%, or at least 3.5 wt.-%, or at least 3.75 wt.-%, or at least 4.0 wt.-%, or at least 4.25 wt.-%, or at least 4.5 wt.-%, or at least 4.75 wt.-%, or at least 5.0 wt.-%, in each case relative to the total weight of the cosmetic composition.

In preferred embodiments of the cosmetic composition according to the invention, the weight content of the pigment component is at most 2.5 wt.-%, or at most 3.0 wt.-%, or at most 3.5 wt.-%, or at most 4.0 wt.-%, or at most 4.5 wt.-%, or at most 5.0 wt.-%, or at most 6.5 wt.-%, or at most 7.0 wt.-%, or at most 7.5 wt.-%, or at most 8.0 wt.-%, or at most 9.0 wt.-%, or at most 10 wt.-%, or at most 12.5 wt.-%, or at most 15 wt.-%, or at most 17.5 wt.-%, or at most 20 wt.-%, or at most 22.5 wt.-%, or at most 25 wt.-%, in each case relative to the total weight of the cosmetic composition.

Preferably, the pigment component comprises or essentially consists of an organic pigment, a mineral pigment, a nacreous pigment, or any combination thereof.

Preferred organic pigments are precipitated onto a substrate, e.g. aluminum hydroxide or calcium hydroxide, thus forming an insoluble salt.

Preferred examples or mineral pigments include but are not limited to titanium oxides, magnesium oxides, cerium oxides, zinc oxides, chromium oxides, and iron oxides (e.g. yellow iron oxide, red iron oxide, black iron oxide).

Preferred examples or nacreous pigments include but are not limited to guanine (2-amino-6-hydroxy-purine), bismuth oxychloride, titanium dioxide/mica or mica coated by several different layers (silica, iron oxide, titanium dioxide, etc.).

Additional powder components that may be contained in the cosmetic composition according to the invention include, but are not limited to, inorganic powders such as talc, gums, chalk, Fuller's earth, sericite, muscovite, phlogopite, synthetic mica, lepidolite, biotite, lithia mica, vermiculite, aluminum silicate, alkyl and/or trialkyl aryl ammonium smectites, chemically modified magnesium aluminum silicate, organically modified montmorillonite clay, hydrated aluminum silicate, fumed aluminum starch, octenyl succinate, barium silicate, calcium silicate, magnesium silicate, strontium silicate, metal tungstate, magnesium, silica alumina, zeolite, barium sulfate, calcined calcium sulfate (calcined gypsum), calcium phosphate, fluorine apa-tite, hydroxyapatite, ceramic powder, colloidal silicone dioxide, and boron nitride; organic powders such as polyamide resin powder (nylon powder), cyclodextrin, methyl polymethacrylate powder, copolymer powder of styrene and acrylic acid, benzoguanamine resin powder, poly(ethylene tetrafluoride) powder, and carboxyvinyl polymer, cellulose powder such as hydroxyethyl cellulose and sodium carboxymethyl cellulose, ethylene glycol monostearate.

The cosmetic composition according to the invention may be available in the form of different product types.

In preferred embodiments, the cosmetic composition according to the invention is a skin or tooth cleansing composition to be rinsed off with water after topical application to the skin or teeth and cleaning.

In preferred embodiments, the cosmetic composition according to the invention is selected from the group consisting of exfoliating scrub soaps, body scrubs, peels (face peels, hand peels, foot peels), handwashing pastes, liquid soaps, shower gels, hair-removal compositions, face masks, and antiacne compositions.

In preferred embodiments, the cosmetic composition according to the invention is a skin care composition to remain on the skin after topical application.

In preferred embodiments, the cosmetic composition according to the invention is selected from hand care composition, body care composition, intimate care compositions, footcare compositions, sunscreen compositions, nailcare compositions, make-up, and repellents.

Another aspect of the invention relates to the use of a cosmetic composition according to the invention as a skin cleansing composition to be rinsed off with water after topical application to the skin and cleaning.

Preferably, the skin cleansing composition is selected from liquid soap, foam soap, shower gel and hair shampoo.

In preferred embodiments, the cosmetic composition according to the invention is used as exfoliating scrub soap, body scrub, peel (face peel, lip peel, hand peel, foot peel), handwashing paste, tooth paste, hair-removal composition, face mask, or antiacne composition and lotions, powders or make-up.

The cosmetic composition according to the invention may be provided in suitable containers that are conventionally used, such as bottles, boxes, packages, dispensers, and the like. The containers can dispense a predetermined amount of the cosmetic composition. In certain aspects, the cosmetic composition is dispensed in a spray, dollop, or liquid.

Another aspect of the invention relates to the use of a cosmetic composition according to the invention as a skin care composition to remain on the skin after topical application.

In preferred embodiments, the cosmetic composition according to the invention is used as hand care composition, body care composition, intimate care composition, footcare composition, tooth paste, sunscreen composition, nailcare composition, make-up or repellent.

### EXAMPLES

The following examples further illustrate the invention but are not to be construed as limiting its scope.

### Body Peeling Oil Gel-Scrub and Lip Scrubs (Example 1 and Example 2):

General preparation procedure: All exemplified cosmetic compositions in Example 1 and Example 2 have been prepared from three phases (A, B and C) with different ingredients. The ingredients of PHASE A have been mixed sequentially and heated to a temperature of 80° C. under continuous stirring until a clear solution was obtained. Subsequently, the solution was allowed to cool down under continuous stirring until a turbid (hazy) solution was obtained. PHASE B was added and homogenized for 2 minutes using a homogenizer. Finally, PHASE C was added and the components were mixed thoroughly and left untouched. All percentages are based on the total content of the ingredients of PHASE A+PHASE B+PHASE C.

### Example 1

Seven comparative body peeling oil gel-scrubs (C1 to C7) and one inventive body peeling oil gel-scrub (I1) were prepared from the following ingredients:

| PHASE | INGREDIENT [wt.-%] | C1 | C2 | C3 | C4 | C5 | C6 | C7 | I1 |
|---|---|---|---|---|---|---|---|---|---|
| A | Coconut oil | 20.0 | 20.0 | 20.0 | 20.0 | 20.0 | 20.0 | 20.0 | 20.0 |
| | Sunflower oil | 14.0 | 14.0 | 14.0 | 14.0 | 14.0 | 14.0 | 14.0 | 14.0 |
| | Avocado oil | 10.0 | 10.0 | 10.0 | 10.0 | 10.0 | 10.0 | 10.0 | 10.0 |
| | Rice bran oil | 10.0 | 10.0 | 10.0 | 10.0 | 10.0 | 10.0 | 10.0 | 10.0 |
| | Castor oil | 10.0 | 10.0 | 10.0 | 10.0 | 10.0 | 10.0 | 10.0 | 10.0 |
| | Grapeseed oil | 10.0 | 10.0 | 10.0 | 10.0 | 10.0 | 10.0 | 10.0 | 10.0 |
| | Hydrogenated vegetable oil | 5.0 | 5.0 | 5.0 | 5.0 | 5.0 | 5.0 | 5.0 | 5.0 |
| | Tocopherol and sunflower | 0.5 | 0.5 | 0.5 | 0.5 | 0.5 | 0.5 | 0.5 | 0.5 |
| | seed oil | | | | | | | | |
| B | Fragrance | 0.5 | 0.5 | 0.5 | 0.5 | 0.5 | 0.5 | 0.5 | 0.5 |
| C | Natural PE beads (500 μm) | 20.0 | — | — | — | — | — | — | — |
| | Shells & Kernels (500 μm) | — | 20.0 | — | — | — | — | — | — |
| | Fruits (500 μm) | — | — | 20.0 | — | — | — | — | — |
| | Seeds (1000 μm) | — | — | — | 20.0 | — | — | — | — |
| | Herbs & Spices (500 μm) | — | — | — | — | 20.0 | — | — | — |
| | Loofah (30 μm) | — | — | — | — | — | 20.0 | — | — |
| | Mineral (500 μm) | — | — | — | — | — | — | 20.0 | — |
| | Cellobiose (D50 150 μm) | — | — | — | — | — | — | — | 20.0 |

### Example 2

Seven comparative lip scrubs (C8 to C14) and one inventive lip scrub (I2) were prepared from the following ingredients:

| PHASE | INGREDIENT [wt.-%] | C8 | C9 | C10 | C11 | C12 | C13 | C14 | I2 |
|---|---|---|---|---|---|---|---|---|---|
| A | Sunflower oil | 15.0 | 15.0 | 15.0 | 15.0 | 15.0 | 15.0 | 15.0 | 15.0 |
| | Grapeseed oil | 15.0 | 15.0 | 15.0 | 15.0 | 15.0 | 15.0 | 15.0 | 15.0 |
| | Avocado oil | 12.0 | 12.0 | 12.0 | 12.0 | 12.0 | 12.0 | 12.0 | 12.0 |
| | Rice bran oil | 12.0 | 12.0 | 12.0 | 12.0 | 12.0 | 12.0 | 12.0 | 12.0 |
| | Castor oil | 12.0 | 12.0 | 12.0 | 12.0 | 12.0 | 12.0 | 12.0 | 12.0 |
| | Hydrogenated vegetable oil | 5.0 | 5.0 | 5.0 | 5.0 | 5.0 | 5.0 | 5.0 | 5.0 |
| | Marula oil | 2.0 | 2.0 | 2.0 | 2.0 | 2.0 | 2.0 | 2.0 | 2.0 |
| | Tocopherol/sunflower seed | 0.5 | 0.5 | 0.5 | 0.5 | 0.5 | 0.5 | 0.5 | 0.5 |
| | oil | | | | | | | | |
| B | Bikira olive/Moringa butter | 6.0 | 6.0 | 6.0 | 6.0 | 6.0 | 6.0 | 6.0 | 6.0 |
| | Fragrance | 0.5 | 0.5 | 0.5 | 0.5 | 0.5 | 0.5 | 0.5 | 0.5 |
| C | Natural PE beads (300 μm) | 20.0 | — | — | — | — | — | — | — |
| | Shells & Kernels (200 μm) | — | 20.0 | — | — | — | — | — | — |
| | Fruits (200 μm) | — | — | 20.0 | — | — | — | — | — |
| | Seeds (1000 μm) | — | — | — | 20.0 | — | — | — | — |
| | Herbs & Spices (200 μm) | — | — | — | — | 20.0 | — | — | — |
| | Loofah (20 μm) | — | — | — | — | — | 20.0 | — | — |
| | Mineral (100 μm) | — | — | — | — | — | — | 20.0 | — |
| | Cellobiose (D50 150 μm) | — | — | — | — | — | — | — | 20.0 |

The cosmetic compositions were tested and evaluated in a panel test with 12 panelists (volunteers). The two different kinds of water-free cosmetic compositions (body peeling oil gel-scrubs of Example 1 and lip scrubs of Example 2) were tested for abrasive action and application performance of cellobiose powders during the sensory test on human skin. Assessment was made by means of a questionnaire.

The test criteria were established by the analyst and were described as follows:
(A) When rinsing off with water: How easy and how fast it is to completely rinse-off the product after application;
(B) After rinsing and pat dry: Which product is preferred in terms of effect on skin/skin feel?

The results of the test are compiled in the following tables:

Criterium (A): Average response of the volunteer after rinsing their skin with water:

| | BODY PEELING | | | | | |
|---|---|---|---|---|---|---|
| When rinsing | OIL GEL-SCRUB | | | LIP SCRUB | | |
| with water | I1 | C1 to C7 | same | I2 | C8 to C14 | same |
| Which one is | 62% | 22% | 16% | 54% | 29% | 17% |
| easier to rinse | | | | | | |
| with water? | | | | | | |
| Which one is | 63% | 21% | 16% | 54% | 29% | 17% |
| faster to rinse | | | | | | |
| with water? | | | | | | |

Criterium (B): Average response of the volunteer to the after rinsing and pat dry the skin area:

| After application | | | | | | |
|---|---|---|---|---|---|---|
| on skin, which one | After rinsing and pat dry | | | | | |
| do you prefer in | BODY PEELING | | | | | |
| terms of effect on | OIL GEL-SCRUB | | | LIP SCRUB | | |
| skin/skin feel? | | I1 | Ci | | I2 | Ci |
| | I1 vs. C1 | 33% | 67% | I2 vs. C8 | 67% | 33% |
| | I1 vs. C2 | 22% | 78% | I2 vs. C9 | 11% | 89% |
| | I1 vs. C3 | 33% | 67% | I2 vs. C10 | 67% | 33% |
| | I1 vs. C4 | 67% | 33% | I2 vs. C11 | 56% | 33% |
| | I1 vs. C5 | 78% | 22% | I2 vs. C12 | 78% | 22% |
| | I1 vs. C6 | 67% | 33% | I2 vs. C13 | 56% | 44% |
| | I1 vs. C7 | 56% | 44% | I2 vs. C14 | 25% | 75% |
| TOTAL VOTES | | 57% | 43% | | 71% | 29% |

As shown by the results given in the table above, the majority of volunteers had a clear preference for the peeling product containing cellobiose, when the they were asked for their favorite product with regard to the effect on skin. In case of the body peeling oil gel-scrub product, the inventive formulation (I1 vs. C1-C7), was preferred in 4 of 7 cases (57%). In case of the lip scrub product, the inventive formulation (I2 vs. C8-C14) was preferred in 5 of 7 cases (71%).

Four comparative scrubs based upon natural PE beads (C1/C8), shells & kernels (C2/C9), fruits (C3/C10) and herbs & spices (C5/C12) were compared against the two inventive scrubs I1 and I2, respectively, during application massage over skin. These results are compiled in the following table (not the same means better):

| Result Yes | C1/C8 | C2/C9 | C3/C10 | C5/C12 |
|---|---|---|---|---|
| Do granules have same feel? | 44% | 22% | 22% | 22% |
| Do granules have same | 33% | 11% | 22% | 22% |
| abrasive/scrubbing effect? | | | | |

It has been shown by the results of the above panel test that the cosmetic compositions according to the invention are advantageous over cosmetic compositions known from the prior art.

Cellobiose powder is easy and quickly to rinse-off and leaves a good skin feel after use based from the volunteer's response in comparison to other scrubbing agents available in the market.

Compared to other scrubs such as kernels or minerals the clear white/light color of the products containing cellobiose can be considered a marketing advantage.

### Liquid Handwash Soaps (Example 3 and Example 4):

General preparation procedure: All exemplified liquid handwash soap compositions were prepared from three different types of liquid handwash soaps A, B and C with clear appearance. For reference purposes, a liquid handwash soap D with creamy white appearance was used. Liquid handwash soaps type A to D are commercially available, e.g. type A as Palmolive® “Aquarium”, type B as Frosch® “Sensitiv Seife”, type C as Balea® “Milde Seife Pflege & Hygiene” and type D as Sagrotan® “Sanft zur Haut Stark gegen Bakterien”.

Composition of the commercially available liquid hand soaps A, B, C and D used for the experiments:

| A: Liquid handwash | B: Liquid handwash | C: Liquid handwash | D: Liquid handwash |
|---|---|---|---|
| soap (clear | soap (clear | soap (creamy white | soap (creamy white |
| appearance) | appearance) | appearance) | appearance) |
| Aqua | Aqua | Aqua | Aqua |
| Sodium Laureth Sulfate | Sodium Laureth Sulfate | Sodium Laureth Sulfate | Sodium Laureth Sulfate |
| Sodium Chloride | Sodium Chloride | Sodium Chloride | Sodium Chloride |
| Parfum | Parfum | Parfum | Parfum |
| Sodium Benzoate | Sodium Benzoate | Sodium Benzoate | Sodium Benzoate |
| Citric Acid | Citric Acid | Citric Acid | Citric Acid |
| Cocamidopropyl | Cocamidopropyl | Cocamidopropyl | |
| Betaine | Betaine | Betaine | |
| | Glycerin | Glycerin | Glycerin |
| Cocamide MEA^{[1]} | | | Cocamide MEA^{[1]} |
| Tetrasodium | | | Tetrasodium EDTA^{[2]} |
| EDTA^{[2]} | | | |
| | Lactic Acid | Lactic Acid | |
| Sodium Salicylate | | | Salicylic Acid |
| | Coco-Glucoside | Coco-Glucoside | |
| | Glyceryl Oleate | Glyceryl Oleate | |
| | | Sodium Hydroxide | Sodium Hydroxide |
| | | Potassium Sorbate | Potassium Sorbate |
| | | Tocopherol | Tocopheryl Acetate |
| CI 17200^{[3]} | | Tetrasodium Glutamate | Styrene/Acrylates Co- |
| CI 42090^{[4]} | | Diacetate | polymer |
| | | Hydrogenated Vegetable | Magnesium Nitrate |
| | | Glycerides Citrate | Magnesium Chloride |
| | | Ascorbyl Palmitate | Methylchloroisothiazolinone |
| | | Lecithin | Methylisothiazolinone |
| | | Benzyl Alcohol | Ammonium Lauryl |
| | | Panthenol | Sulfate |
| | | | Aloe Barbadensis |
| | | | Leaf Juice |

^{[1]}MEA = Monoethanolamine;
^{[2]}EDTA = Ethylenediaminetetraacetic acid;
^{[3]}CI 17200 = Disodium 5-amino-4-hydroxy-3-(phenylazo)naphthalene-2,7-disulphonate;
^{[4]}CI 42090 = Dihydrogen (ethyl)[4-[4-[ethyl(3-sulphonatobenzyl)]amino]-2′-sulphonatobenzhydrylidene]cyclohexa-2,5-dien-1-ylidene](3-sulphonatobenzyl)ammonium, disodium salt

### Example 3

To 20 g of liquid handwash soap A, B and C, respectively, 2 g of cellobiose was added. Subsequently, the mixture was stirred at room temperature for 2.5 h using a magnetic stirrer. Liquid handwash soap D was used as a reference example for a liquid handwash soap with creamy white appearance. FIG. 1 shows that liquid handwash soaps A, B and C which have been mixed with 2.0 g cellobiose have a brighter and more homogenous looking creamy white appearance than reference example liquid handwash soap D.

### Example 4

General preparation procedure: Overall, 10 samples have been prepared: 5 samples of commercial liquid handwash soap A and 5 samples of commercial liquid handwash soap B have been prepared. Therefore, for each of the test series with the liquid handwash soaps A and B, respectively, a quantity of five times 20 g of the liquid handwash soap was filled into plastic beakers equipped with screw caps. Subsequently, a different amount of cellobiose (1 g; 1.5 g; 2.0 g; 2.5 g; and 3.0 g) was then added to each, the beaker was sealed, and the mixtures were stirred for 2.5 h at room temperature with a magnetic stirrer. Following, all samples were kept in the sealed beakers for a period of 1 day to 3 days at room temperature. Further, after 1 day and 3 days, respectively, a 1 mL sample was taken of each mixture, trans-ferred to a centrifuge tube and centrifuged at 14.000×g for 30 min.

Samples of commercially available liquid handwash soaps A and B with different amounts of cellobiose added:

| | SAMPLE | | | | | | | | | |
|---|---|---|---|---|---|---|---|---|---|---|
| INGREDIENT [g] | A1 | A2 | A3 | A4 | A5 | B1 | B2 | B3 | B4 | B5 |
| Liquid handwash | 20.0 | 20.0 | 20.0 | 20.0 | 20.0 | — | — | — | — | — |
| soap A | | | | | | | | | | |
| Liquid handwash | — | — | — | — | — | 20.0 | 20.0 | 20.0 | 20.0 | 20.0 |
| soap B | | | | | | | | | | |
| Cellobiose | 1 | 1.5 | 2.0 | 2.5 | 3.0 | 1 | 1.5 | 2.0 | 2.5 | 3.0 |
| Total | 21.0 | 21.5 | 22.0 | 22.5 | 23.0 | 21.0 | 21.5 | 22.0 | 22.5 | 23.0 |
| Weight-% of | 4.8 | 7.0 | 9.1 | 11.1 | 13.0 | 4.8 | 7.0 | 9.1 | 11.1 | 13.0 |
| cellobiose^{[5]} | | | | | | | | | | |

^{[5]}With respect to the total weight of the cosmetic composition.

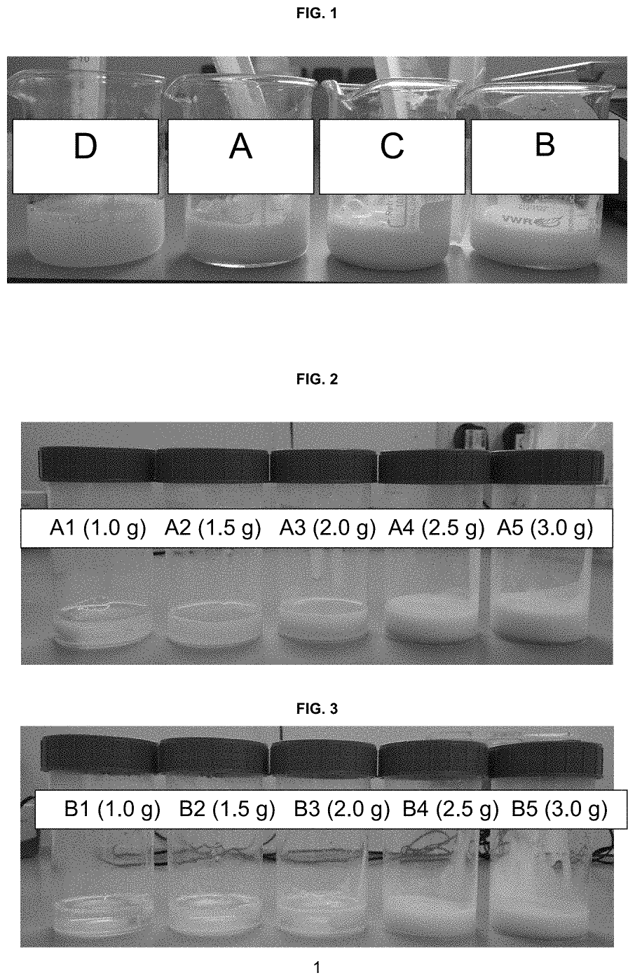

FIGS. 2 and 3 show photographs of samples A1 to A5 after 2.5 h (FIG. 2) and 3 days (FIG. 3) of stirring.

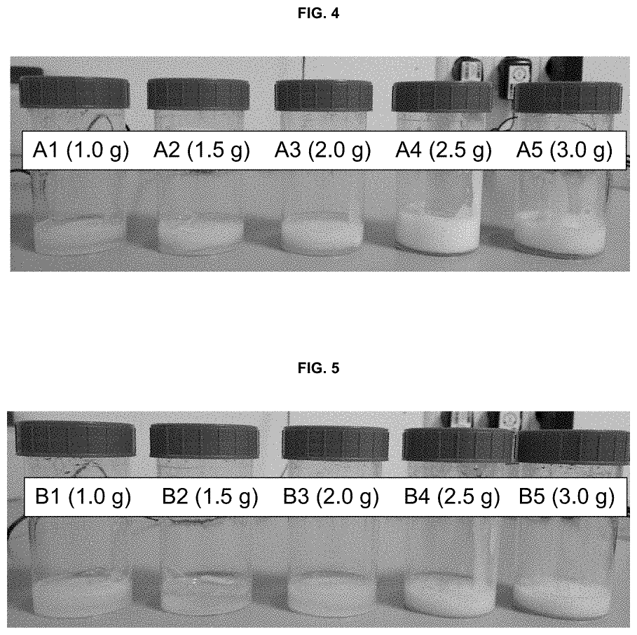

FIGS. 4 and 5 show photographs of samples B1 to B5 after 2.5 h (FIG. 4) and 3 days (FIG. 5) of stirring

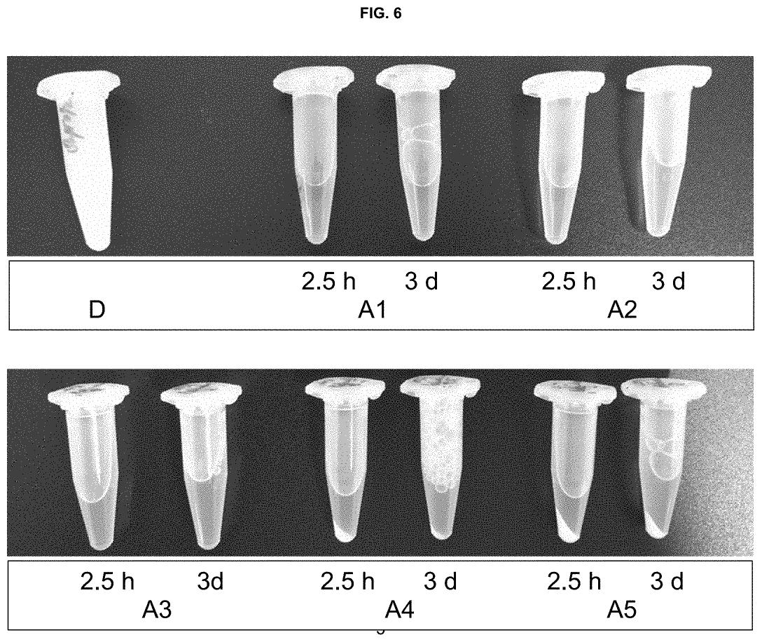

FIG. 6 shows photographs of centrifuged samples A1 to A5 and reference liquid handwash soap D.

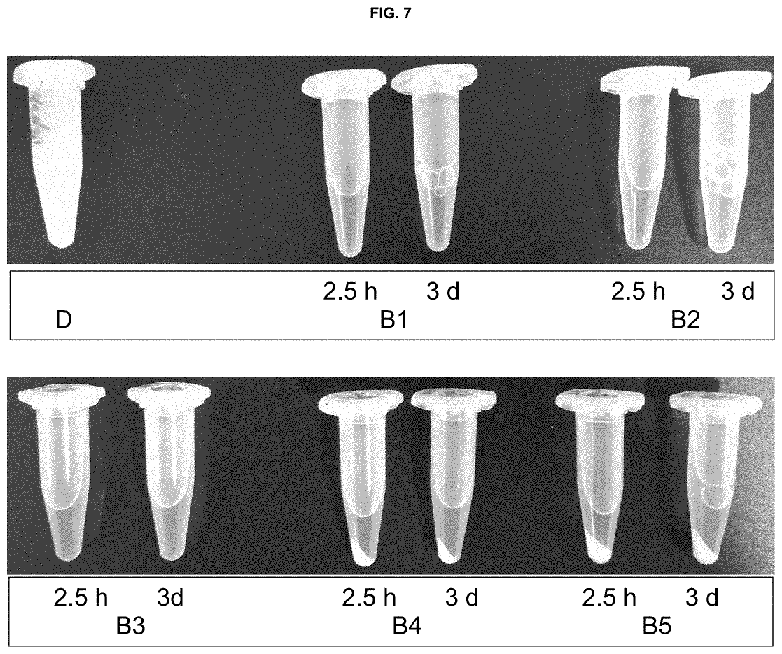

FIG. 7 shows photographs of centrifuged samples B1 to B5 and reference liquid handwash soap D.

It has been shown by the results of the above Example 3 and Example 4 that the cosmetic compositions according to the invention are advantageous over cosmetic compositions known from the prior art, e.g. the liquid handwash soaps with cellobiose have a brighter and more homogenous looking creamy white appearance than comparable liquid handwash soaps known from the prior art.

The project leading to this patent application has received funding from the European Union's Horizon 2020 research and innovation program under grant agreement No. 761030.

1. A cosmetic composition comprising a cellodextrin component comprising a cellosaccharide selected from the group consisting of cellobiose, cellotriose, cellotetraose, cellopentaose, cellohexaose, celloheptaose, cellooctaose, cellononaose, cellodecaose, celloundecaose, cellododecaose, and combinations thereof;
(i) a lipoid component comprising one or more natural or synthetic lipoids; and/or
(ii) a surfactant component comprising an anionic surfactant, cationic surfactant, amphoteric surfactant, nonionic surfactant, or any combination thereof; and/or
(iii) a thickening component; and/or
(iv) a pigment component;
wherein at least a portion of said cellodextrin component is present within the cosmetic composition in undissolved form;
optionally wherein a total content of the cellodextrin component is at least 0.01 wt.-%, relative to a total weight of the cosmetic composition.

2. (canceled)

3. (canceled)

4. (canceled)

5. The cosmetic composition according to claim 1, wherein the cosmetic composition comprises a multitude of solid particles, wherein at least a portion of the multitude of solid particles comprises at least a portion of the cellodextrin component.

6. (canceled)

7. (canceled)

8. (canceled)

9. (canceled)

10. The cosmetic composition according to claim 5, wherein the particles within the multitude of solid particles (i.e. the cellodextrin-containing particles)
have a D50(v) value of at most 100 μm, or at most 50 μm, or at most 10 μm, or at most 5.0 μm, or at most 1.0 μm, or at most 0.5 μm, or at most 0.1 μm, or at most 0.05 μm, or at most 0.01 μm, or at most 0.005 μm, or at most 0.001 μm, determined by laser diffraction analysis according to ISO 13320; or
exhibit a span of at least 0.5, determined by laser diffraction analysis according to ISO 13 320.

11. The cosmetic composition according to claim 5, wherein the particles within the multitude of solid particles (i.e. the cellodextrin-containing particles) have a
D10(v) value within the range of from 0.1 μm to 2 500 μm; preferably from 1 000 μm to 2 000 μm, or from 500 μm to 1 000 μm, or from 200 μm to 500 μm, or from 75 μm to 200 μm, or from 0.1 μm to 4 μm, or from 0.3 μm to 1.0 μm, or from 0.3 μm to 0.8 μm; and/or
D50(v) value within the range of from 0.1 μm to 2 500 μm; preferably from 1 000 μm to 2 000 μm, or from 500 μm to 1000 μm, or from 200 μm to 500 μm, or from 75 μm to 200 μm, or from 0.1 μm to 4 μm, or from 0.3 μm to 1.0 μm, or from 0.3 μm to 0.8 μm; and/or
D90(v) value within the range of from 0.1 μm to 2500 μm; preferably from 1 000 μm to 2 000 μm, or from 500 μm to 1 000 μm, or from 200 μm to 500 μm, or from 75 μm to 200 μm, or from 0.1 μm to 4 μm, or from 0.3 μm to 1.0 μm, or from 0.3 μm to 0.8 μm;
determined by laser diffraction analysis according to ISO 13320.

12. (canceled)

13. (canceled)

14. (canceled)

15. The cosmetic composition according to claim 5, wherein
at least 10 wt.-%, or at least 20 wt.-%, or at least 30 wt.-%, or at least 40 wt.-%, or at least 50 wt.-%, or at least 60 wt.-%, or at least 70 wt.-%, or at least 80 wt.-%, or at least 90 wt.-%; and/or
at most 90 wt.-%, or at most 80 wt.-%, or at most 70 wt.-%, or at most 60 wt.-%, or at most 50 wt.-%, or at most 40 wt.-%, or at most 30 wt.-%, or at most 20 wt.-%, or at most 10 wt.-%
of the particles within the multitude of solid particles (i.e. the cellodextrin-containing particles) have an average particle size, preferably expressed as D50(v) value, within the range of from 0.1 μm to 2 000 μm, determined by laser diffraction analysis according to ISO 13 320.

16. (canceled)

17. (canceled)

18. (canceled)

19. (canceled)

20. The cosmetic composition according to claim 5, wherein at least 10 wt.-%, or at least 20 wt.-%, or at least 30 wt.-%, or at least 40 wt.-%, or at least 50 wt.-%, or at least 60 wt.-%, or at least 70 wt.-%, or at least 80 wt.-%, or at least 90 wt.-% of the particles within the multitude of solid particles (i.e. the cellodextrin-containing particles) have an average particle size, preferably expressed as D50(v) value, within the range
(i) of from 0.1 μm to 4.0 μm; or
(ii) of from 0.2 μm to 2.0 μm; or
(iii) of from 0.3 μm to 1.0 μm; or
(iv) of from 0.3 μm to 0.8 μm;
determined by laser diffraction analysis according to ISO 13320.

21. (canceled)

22. The cosmetic composition according to claim 1, wherein the weight content of the lipoid component is at least 5 wt.-%, or at least 10 wt.-%, or at least 15 wt.-%, or at least 20 wt.-%, or at least 25 wt.-%, or at least 30 wt.-%, or at least 35 wt.-%, or at least 40 wt.-%, or at least 45 wt.-%, or at least 50 wt.-%, or at least 55 wt.-%, or at least 60 wt.-%, or at least 65 wt.-%, in each case relative to the total weight of the cosmetic composition.

23. (canceled)

24. The cosmetic composition according to claim 1, wherein the lipoid component comprises or essentially consists of
(i) a natural lipoid from plant origin, preferably selected from the group consisting of amaranth oil, apricot kernel oil, argan oil, avocado oil, acai oil, babassu oil, baobab oil, bayberry wax, black cumin oil, black currant seed oil, borage seed oil, brazil nut oil, broccoli seed oil, cacao butter, calendula oil, camelina oil, camellia seed oil, carrot seed oil, castor oil, cedarwood seed oil, chamomile oil, chia oil, coconut oil, corn oil, cottonseed oil, cupuacu butter, cranberry seed oil, eucalyptus oil, evening primrose oil, frankincense oil, geranium oil, grape seed oil, groundnut oil, hazelnut oil, hemp oil, illipe butter, Japan wax, jasmine oil, jojoba oil, kokum butter, kukui nut oil, laurel oil, lavender oil, lemongrass oil, linseed oil, macadamia nut oil, mango butter, manketti oil, marula oil, meadowfoam seed oil, milk butter, milk thistle oil, moringa oil, mustard seed oil, olive oil, olus oil, palm oil, palm kernel oil, papaya seed oil, passionfruit seed oil, peach kernel oil, peanut oil, pecan nut oil, peppermint oil, perilla oil, pistachio nut oil, plum kernel oil, pomegranate oil, poppyseed oil, pumpkin seed oil, rapeseed oil, raspberry seed oil, rice bran oil, rose oil, rosehip oil, rosemary oil, sacha inchi oil, safflower oil, sea buckthorn pulp oil, sesame oil, shea butter, soybean oil, St. John's wort oil, sunflower seed oil, squalane oil, sweet almond oil, tall oil, tamanu oil, tea-tree oil, tigernut oil, thyme oil, tung oil, walnut oil, wheat germ oil, and combinations thereof; or
(ii) a natural lipoid from animal origin, preferably selected from the group consisting of fish oil, whale oil, fish liver oil, seal oil, and combinations thereof; or
(iii) a synthetic oil based upon fuel, preferably selected from the group consisting of white mineral oil, paraffin oil, petroleum jelly oil, petrolatum, and combinations thereof; or
(iv) a synthetic fat based upon fuel, preferably paraffin;
(v) a synthetic silicone oil, preferably selected from the group consisting of dimethylpolysiloxane, cyclic silicones, methylphenylpolysiloxane, silicone-glycol copolymers, and combinations thereof;
(vi) a synthetic silicone fat;
(vii) a biochemically synthesized oil;
(viii) a biochemically synthesized fat; or any combination of the foregoing.

25. The cosmetic composition according to claim 24, wherein the lipoid component comprises or essentially consists of a combination of at least two, or at least three, or at least four, or at least five, or at least six of said natural lipoids, preferably from plant origin.

26. (canceled)

27. (canceled)

28. (canceled)

29. (canceled)

30. The cosmetic composition according to claim 1, which is a water-in-oil emulsion or an oil-in-water emulsion.

31. (canceled)

32. The cosmetic composition according to claim 1, wherein the weight content of the surfactant component is at least 0.1 wt.-%, or at least 1.0 wt.-%, or at least 2.0 wt.-%, or at least 4.0 wt.-%, or at least 6.0 wt.-%, or at least 8.0 wt.-%, or at least 10 wt.-%, or at least 15 wt.-%, or at least 20 wt.-%, or at least 25 wt.-%, or at least 30 wt.-%, or at least 35 wt.-%, or at least 40 wt.-%, or at least 45 wt.-%, or at least 50 wt.-%, or at least 55 wt.-%, or at least 60 wt.-%, or at least 65 wt.-%, or at least 70 wt.-%, or at least 75 wt.-%, or at least 80 wt.-%, or at least 85 wt.-%, or at least 90 wt.-%, or at least 95 wt.-%, in each case relative to the total weight of the cosmetic composition.

33. (canceled)

34. The cosmetic composition according to claim 1, wherein the surfactant component comprises or essentially consists of a surfactant selected from the group consisting of
anionic surfactants which are preferably selected from: sodium laureth sulfate, sodium dodecyl sulfate, sodium lauryl sulfate, ammonium lauryl sulfate, sulfosuccinates (e.g. dioctyl sulfosuccinate, disodium laureth sulfosuccinate, disodium PEG-5 laurylcitrate sulfosuccinate, diethylhexyl sodium sulfosuccinate, disodium ricinoleamido MEA-sulfosuccinate, disodium undecylenamido MEA-sulfosuccinate), acyl methyl taurates (e.g. sodium N-methyl lauroyl taurate), alkylbenzene sulfonates (e.g. dodecylbenzene sulfonate), acyl sarcosinates (e.g. sodium lauroyl sarcosinate), fatty glycerol ether sulfonates (e.g. sodium (3-lauryloxy)-2-hydroxypropane sulfonate), acyl isethionate (e.g. sodium lauroly isethionate), monoglyceride sulfates (e.g. sodium (3-lauroyloxy)-2-hydroxypropyl sulfate), propyl peptide condensates;
cationic surfactants which are preferably selected from: stearalkonium chlorides (benzyldimethyloctadecylammonium chloride), dicetyldimonium chloride, docosyltrimethylammonium chloride;
amphoteric surfactants which are preferably selected from: sodium lauraminopropionate, sodium cocoamphopropionate, cocamidopropyl betaines (e.g. lauramidopropyl betaine), capryl/capramidopropyl betaine, undecylenamidopropyl betaine, disodium cocoamphodiacetate, sodium cocoamphoacetate;
nonionic surfactants which are preferably selected from: lauramide, lauramide diethanolamine, lauramide oxide, stearamine oxide, poloxamers (e.g. Poloxamer 101, Poloxamer 124), PEG-7 glyceryl cocoate, glyceryl laurate, cocamide diethanolamine, cocamide monoethanolamine;
biosurfactants which are preferably selected from (i) glycolipids (carbohydrate-lipids) such as rhamnolipids, mannosylerythritol lipids, sophorolipids, cellobioselipids, and the like; (ii) phospholipids; (iii) polyol lipids; (iv) lipoproteins; (v) lipopeptides; (vi) ornithine lipids; (vii) neutral lipids; (viii) aminoacid lipids; (ix) exolipids; (x) liposan; (xi) siderolipids; (xii) protein polyamines diglycosyl diglycerides; (xiii) fimbriae; and combinations thereof.

35. The cosmetic composition according to claim 5, wherein
at least 50 wt.-%, preferably all of the particles within the multitude of solid particles (i.e. the cellodextrin-containing particles) have an average particle size, preferably expressed as D50(v) value, within the range of from 0.1 μm to 4.0 μm, or from 0.2 μm to 2.0 μm, or from 0.3 μm to 1.0 μm, or from 0.3 μm to 0.8 μm, determined by laser diffraction analysis according to ISO 13320; and/or
the weight content of the surfactant component is at least 5.0 wt.-%, preferably at least 10 wt.-%, relative to the total weight of the cosmetic composition; and/or
the cosmetic composition additionally comprises water, wherein the water content is at least 80 wt.-%, relative to the total weight of the cosmetic composition.

36. The cosmetic composition according to claim 1, wherein
the cellodextrin component comprises or essentially consists of cellobiose;
the cosmetic composition comprises solid particles comprising or essentially consisting of the cellodextrin component in undissolved form;
the particles have an average particle size, preferably expressed as D50(v) value determined by laser diffraction analysis according to ISO 13320, of at most 100 μm, or at most 50 μm, or at most 10 μm, or at most 5.0 μm, or at most 1.0 μm, or at most 0.5 μm, or at most 0.1 μm, or at most 0.05 μm, or at most 0.01 μm, or at most 0.005 μm, or at most 0.001 μm;
the weight content of the surfactant component is at least 0.1 wt.-%, preferably at least 1.0 wt.-%, more preferably at least 5.0 wt.-%, still more preferably at least 10 wt.-%, relative to the total weight of the cosmetic composition;
the cosmetic composition additionally comprises water, wherein the water content is at least 20 wt.-%, preferably at least 40 wt.-%, more preferably at least 60 wt.-%, still more preferably at least 80 wt.-%, relative to the total weight of the cosmetic composition;
the cosmetic composition is selected from liquid soap, foam soap, shower gel and hair shampoo.

37. The cosmetic composition according to claim 5, wherein
at least 50 wt.-%, preferably all of the particles within the multitude of solid particles (i.e. the cellodextrin-containing particles) have an average particle size, preferably expressed as D50(v) value, within the range of from 1 000 μm to 2 000 μm, or from 500 μm to 1 000 μm, or from 200 μm to 500 μm, or from 75 μm to 200 μm, determined by laser diffraction analysis according to ISO 13320; and/or
the weight content of the lipoid component is at least 50 wt.-%, preferably 60 wt.-%; and/or
the total content of the cellodextrin component is at least 20 wt.-%; and/or
the portion of the cellodextrin component that is present in undissolved form amounts to at least 95 wt.-%, relative to the total amount of the cellodextrin component that is contained in the cosmetic composition; and/or
the cosmetic composition comprises essentially no water.

38. The cosmetic composition according to claim 1, wherein
the cellodextrin component comprises or essentially consists of cellobiose;
the cosmetic composition comprises solid particles comprising or essentially consisting of the cellodextrin component in undissolved form;
the particles have an average particle size, preferably expressed as D50(v) value determined by laser diffraction analysis according to ISO 13320, of at least 0.001 μm, or at least 0.005 μm, or at least 0.01 μm, or at least 0.05 μm, or at least 0.1 μm, or at least 0.5 μm, or at least 1.0 μm, or at least 5.0 μm, or at least 10 μm, or at least 50 μm, or at least 100 μm;
the weight content of the lipoid component is at least 0.1 wt.-%, preferably at least 1.0 wt.-%, more preferably at least 5.0 wt.-%, still more preferably at least 10 wt.-%, yet more preferably at least 20 wt.-%, even more preferably at least 30 wt.-%, most preferably at least 40 wt.-%, and in particular at least 50 wt.-%, relative to the total weight of the cosmetic composition;
the cosmetic composition comprises essentially no water, or wherein the water content is at most 20 wt.-%, preferably at most 15 wt.-%, more preferably at most 10 wt.-%, still more preferably at most 5.0 wt.-%, relative to the total weight of the cosmetic composition; and
the cosmetic composition is selected from the group consisting of exfoliating scrub soaps, body scrubs, peels (face peels, hand peels, foot peels), handwashing pastes, liquid soaps, shower gels, hair-removal compositions, face masks, and antiacne compositions.

39. The cosmetic composition according to claim 1, wherein the cellodextrin component comprises or essentially consists of cellobiose,
optionally wherein the weight content of the cellobiose is at least 1.0 wt.-%, relative to the total weight of the cosmetic composition.

40. (canceled)

41. (canceled)

42. (canceled)

43. (canceled)

44. (canceled)

45. (canceled)

46. (canceled)

47. (canceled)

48. The cosmetic composition according to claim 1,
which has an opacity of at least 25%, determined by colorimeter measurements; or
which has an opacity of at most 95%, determined by colorimeter measurements; or
which has a turbidity of at least 5 NTU determined by nephelometric analysis; or
which has a turbidity of at most 900 NTU determined by nephelometric analysis; or
which has a turbidity of at most 900 NTU determined by nephelometric analysis; or
which has an intensity of pearlescence, on a scale of 0 to 10, of at least 1, determined in a sensory test according to ISO 13299; or
which has a dynamic viscosity of at least 10 mPa·s, determined according to ISO 3219; or
which additionally comprises an antioxidant, optionally wherein the antioxidant is a tocopherol.

49. (canceled)

50. (canceled)

51. (canceled)

52. (canceled)

53. (canceled)

54. (canceled)

55. (canceled)

56. (canceled)

57. The cosmetic composition according to claim 1, wherein the thickening agent comprises
(i) a natural or synthetic gum, preferably selected from the group consisting of algin, sodium alginate, carrageenan, cellulose gum, guar gum, xanthan gum, karaya gum, gelatin, tragacanth gum, chitosan, acacia gum, and any combination thereof;
(ii) a cellulose ether, preferably selected from the group consisting of methylcellulose, ethylcellulose, hydroxyethyl cellulose, hydroxypropyl cellulose, hydroxypropyl methyl cellulose, carboxymethyl cellulose, and any combination thereof;
(iii) a carbomer (e.g., a crosslinked polyacrylic acid copolymer or homopolymer and copolymers of acrylic acid cross linked with a polyalkenyl polyether);
(iv) an acrylamide polymer, acrylic acid polymer or vinyl polymer, preferably selected from polyvinyl alcohol and polyvinylpyrrolidone;
(v) a polyamine or polyquaternary compound;
(vi) an ethylene oxide polymer;
or any mixtures thereof.

58. The cosmetic composition of claim 1, which is
a skin cleaning composition to be rinsed off with water after topical application to the skin and cleaning; or
a skin care composition to remain on the skin after topical application.

59. (canceled)

60. (canceled)

61. (canceled)

62. (canceled)

63. (canceled)

64. (canceled)

65. (canceled)

66. (canceled)
